(19) United States
(12) Patent Application Publication
(10) Pub. No.: US 2014/0137463 A1
(43) Pub. Date: May 22, 2014

(54) Low Polysaccharide Microorganisms for Production of Biofuels and Other Renewable Materials

(71) Applicants: Kirk Apt, Ellicott City, MD (US); Jacob Borden, San Diego, CA (US); Paul W. Behrens, Warrenville, IL (US); David Dain, Baltimore, MD (US); Jon Hansen, West Friendship, MD (US); Joseph W. Pfeifer, Westminister, MD (US)

(72) Inventors: Kirk Apt, Ellicott City, MD (US); Jacob Borden, San Diego, CA (US); Paul W. Behrens, Warrenville, IL (US); David Dain, Baltimore, MD (US); Jon Hansen, West Friendship, MD (US); Joseph W. Pfeifer, Westminister, MD (US)

(73) Assignee: BP Corporation North America Inc., Naperville, IL (US)

(21) Appl. No.: 13/859,229

(22) Filed: Apr. 9, 2013

Related U.S. Application Data
(60) Provisional application No. 61/621,761, filed on Apr. 9, 2012.

(51) Int. Cl. C12P 7/64; C10L 1/18; F02B 43/00; C12R 1/645

(52) U.S. Cl. CPC C12P 7/6409; C12R 1/645; C10L 1/1802; F02B 43/00
USPC 44/307; 435/254.2; 435/134; 123/1 A

(57) ABSTRACT

High cell density fermentations of wild-type organisms can result in increased viscosity due to the production of exocellular polysaccharides. Mutant microorganisms with a dry morphology, resulting from reduced exocellular polysaccharide formation, were isolated and characterized. The exocellular polysaccharide composition for these modified microorganisms is shown to be different than the polysaccharide composition of the wild type microorganism. In addition to reduced exocellular polysaccharide formation, dry morphology mutants of multiple strains show reduced viscosity, improved oxygen mass transfer, and improved fatty acid fermentation yield on carbon.

This application claims the benefit of U.S. Provisional Patent Application Ser. No. 61/621,761 filed on Apr. 9, 2002, which is hereby incorporated herein by reference in its entirety.

### BACKGROUND OF THE INVENTION

### Names of the Parties to a Joint Research Agreement

For purposes of 35 U.S.C. §103(c)(2), a joint research agreement was executed between BP Biofuels UK Limited and Martek Biosciences Corporation on Dec. 18, 2008 in the field of renewable materials. Also for the purposes of 35 U.S.C. §103(c)(2), a joint development agreement was executed between BP Biofuels UK Limited and Martek Biosciences Corporation on Aug. 7, 2009 in the field of renewable materials. Also for the purposes of 35 U.S.C. §103(c)(2), a joint development agreement was executed between BP Biofuels UK Limited and DSM Biobased Products and Services B.V. on Sep. 1, 2012 in the field of renewable materials.

### TECHNICAL FIELD

This application is directed to microorganisms, media, biological oils, biofuels, and/or methods suitable for use in lipid production.

### BACKGROUND

Issues of greenhouse gas levels and climate change have led to development of technologies seeking to utilize natural cycles between fixed carbon and liberated carbon dioxide. As these technologies advance, various techniques to convert feedstocks into biofuels have been developed. However, even with the above advances in technology, there remains a need and a desire to improve economic viability for conversion of renewable carbon sources to fuels.

Biodiesel fuel has clear benefits being renewable, biodegradable, nontoxic, and containing neither sulfur nor aromatics. But one of its disadvantages is high cost, most of which is due to the cost of vegetable oil. Therefore, the economic aspect of biodiesel fuel production has been restricted by the cost of oil raw materials, such as lipids.

Lipids for use in biofuels and other renewable materials can be produced in microorganisms, such as yeast, algae, fungi, or bacteria. Manufacturing a lipid in a microorganism involves growing microorganisms which are capable of producing a desired lipid in a fermentor or bioreactor, isolating the microbial biomass, drying it, and extracting the intracellular lipids. However, biofuel and other renewable material applications require high density fermentations, and many microorganisms cannot reach high levels of cell density fermentation due to increased media viscosity, and are thus not suited for high cell density applications, such as biofuels and other renewable materials.

There is a need for microorganisms for production of biofuels and other renewable materials that produce fermentation broth with low viscosity and a high mass transfer coefficient to support high cell density levels.

### BRIEF DESCRIPTION OF THE DRAWINGS

The accompanying drawings, which are incorporated in and constitute a part of this specification, illustrate embodiments of the disclosure and, together with the description, serve to explain the features, advantages, and principles of the disclosure. In the drawings:

FIG. 1: Graph showing the decrease in power per volume (P/V) from 2000 as viscosity of the solution increases. The figure depicts both low and high oxygen transfer conditions.

FIG. 2: Graph of P/V needed to deliver oxygen to the solution according to the viscosity of the solution. The figure depicts both low and high oxygen transfer conditions.

FIG. 3: Graph of solution viscosity as a function of polysaccharide concentration in grams per liter.

FIG. 4: Representative result from ion-exchange chromatography (IEC) of acid hydrolyzed polysaccharide of wild type (abbreviated “WT”) strain MK29404

FIG. 5: Representative result from ion-exchange chromatography (IEC) analysis of acid hydrolyzed polysaccharide of mutant MK29404 Dry-1 strain

FIG. 6: Representative result from size exclusion chromatography of isolated polysaccharides.

### DETAILED DESCRIPTION OF EMBODIMENTS

Production of oils from microorganisms has many advantages over production of oils from plants, such as short life cycle, less labor requirement, independence of season and climate, and easier scale-up. Cultivation of microorganisms also does not require large acreages and there is no competition with food production.

High cell density fermentations of wild type (abbreviated “WT”) organisms can result in increased viscosity due to the production of exocellular polysaccharides triggered by the same nitrogen limiting conditions that facilitate lipid production. Mutants with a “dry” morphology and/or phenotype, indicating reduced polysaccharide formation, were isolated and characterized. In addition to reduced polysaccharide formation, dry phenotype mutants of multiple strains can also exhibit reduced viscosity, improved oxygen mass transfer, improved fermentation yield on carbon, and improved lipid extractability.

The disclosure relates to oil-producing microorganisms and to methods of cultivating such microorganisms for the production of useful compounds, including lipids, fatty acid esters, fatty acids, aldehydes, alcohols, alkanes, fuels, fuel and precursors, for use in industry and fuels, or as an energy and food sources. The microorganisms as disclosed in the application can be selected or genetically engineered for use in the methods or other aspects of the according to the disclosure described herein.

### 1. DEFINITIONS

Unless defined otherwise, all technical and scientific terms used herein have the meaning commonly understood by a person skilled in the art to which this disclosure belongs. The following references provide one of skill with a general definition of many of the terms used in this disclosure: Singleton at al., Dictionary of Microbiology and Molecular Biology (2nd ed. 1994); The Cambridge Dictionary of Science and Technology (Walker ed., 1988); The Glossary of Genetics, 5th Ed., R. Rieger et al., eds., Springer Verlag (1991); Hale & Marham, The Harper Collins Dictionary of Biology (1991); Sambrook at al., Molecular Cloning: A Laboratory Manual, (3d edition, 2001, Cold Spring Harbor Press).

As used herein, the following terms have the meanings ascribed to them unless specified otherwise.

As used herein, the terms “has,” “having,” “comprising,” “with,” “containing,” and “including” are open and inclusive expressions. Alternately, the term “consisting” is a closed and exclusive expression. Should any ambiguity exist in construing any term in the claims or the specification, the intent of the drafter is toward open and inclusive expressions.

As used herein, the term “and/or the like” provides support for any and all individual and combinations of items and/or members in a list, as well as support for equivalents of individual and combinations of items and/or members.

Regarding an order, number, sequence, omission, and/or limit of repetition for steps in a method or process, the drafter intends no implied order, number, sequence, omission, and/or limit of repetition for the steps to the scope of the invention, unless explicitly provided.

Regarding ranges, ranges are to be construed as including all points between upper values and lower values, such as to provide support for all possible ranges contained between the upper values and the lower values including ranges with no upper bound and/or lower bound.

Basis for operations, percentages, and procedures can be on any suitable basis, such as a mass basis, a volume basis, a mole basis, and/or the like. If a basis is not specified, a mass basis or other appropriate basis should be used.

The term “substantially,” as used herein, refers to being largely that which is specified and/or identified.

The term “similar,” as used herein, refers to having characteristics in common, such as not dramatically different.

It will be apparent to those skilled in the art that various modifications and variations can be made in the disclosed structures and methods without departing from the scope or spirit of the invention. Particularly, descriptions of any of the embodiments can be freely combined with descriptions of other embodiments to result in combinations and/or variations of two or more elements and/or limitations. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

The terms “producing” and “production,” as used herein, refer to making, forming, creating, shaping, bringing about, bringing into existence, manufacturing, growing, synthesizing, and/or the like. According to some embodiments, producing includes fermentation, cell culturing, and/or the like. Producing can include new or additional organisms as well as maturation of existing organisms.

The term “growing,” as used herein, refers to increasing in size, such as by assimilation of material into the living organism and/or the like.

The term “biological,” as used herein, refers to life systems, living processes, organisms that are alive, and/or the like. Biological can refer to organisms from archaea, bacteria, and/or eukarya. Biological can also refer to derived and/or modified compounds and/or materials from biological organisms. According to some embodiments, biological excludes fossilized and/or ancient materials, such as those whose life ended at least about 1,000 years ago.

The term “oil,” as used herein, refers to hydrocarbon substances and/or materials that are at least somewhat hydrophobic and/or water repelling. Oil can include mineral oil, organic oil, synthetic oil, essential oil, and/or the like. Mineral oil refers to petroleum and/or related substances derived at least in part from the Earth and/or underground, such as fossil fuels. “Organic oil” refers to materials and/or substances derived at least in part from plants, animals, other organisms, and/or the like. “Synthetic oil” refers to materials and/or substances derived at least in part from chemical reactions and/or processes, such as can be used in lubricating oil. Oil can be at least generally soluble in nonpolar solvents and other hydrocarbons, but at least generally insoluble in water and/or aqueous solutions. Oil can be at least about 50 percent soluble in nonpolar solvents, at least about 75 percent soluble in nonpolar solvents, at least about 90 percent soluble in nonpolar solvents, completely soluble in nonpolar solvents, about 50 percent soluble in nonpolar solvents to about 100 percent soluble in nonpolar solvents and/or the like, all on a mass basis.

The term “biological oils,” as used herein, refers to hydrocarbon materials and/or substances derived at least in part from living organisms, such as animals, plants, fungi, yeasts, algae, microalgae, bacteria, and/or the like. According to some embodiments, biological oils can be suitable for use as and/or conversion into biofuels and/or renewable materials. These renewable materials can be used in the manufacture of a food, dietary supplement, cosmetic, or pharmaceutical composition for a non-human animal or human.

The term “lipid,” as used herein, refers to oils, fats, waxes, greases, cholesterol, glycerides, steroids, phosphatides, cerebrosides, fatty acids, fatty acid related compounds, derived compounds, other oily substances, and/or the like. Lipids can be made in living cells and can have a relatively high carbon content and a relatively high hydrogen content with a relatively lower oxygen content. Lipids typically include a relatively high energy content, such as on a mass basis.

The term “renewable materials,” as used herein, refers to substances and/or items that have been at least partially derived from a source and/or process capable of being replaced by natural ecological cycles and/or resources. Renewable materials can include chemicals, chemical intermediates, solvents, monomers, oligomers, polymers, biofuels, biofuel intermediates, biogasoline, biogasoline blendstocks, biodiesel, green diesel, renewable diesel, biodiesel blend stocks, biodistillates, biological oils, and/or the like. In some embodiments, the renewable material can be derived from a living organism, such as plants, algae, bacteria, fungi, and/or the like.

The term “biofuel,” as used herein, refers to components and/or streams suitable for use as a fuel and/or a combustion source derived at least in part from renewable sources. The biofuel can be sustainably produced and/or have reduced and/or no net carbon emissions to the atmosphere, such as when compared to fossil fuels. According to some embodiments, renewable sources can exclude materials mined or drilled, such as from the underground. In some embodiments, renewable resources can include single cell organisms, multicell organisms, plants, fungi, bacteria, algae, cultivated crops, noncultivated crops, timber, and/or the like. Biofuels can be suitable for use as transportation fuels, such as for use in land vehicles, marine vehicles, aviation vehicles, and/or the like. Biofuels can be suitable for use in power generation, such as raising steam, exchanging energy with a suitable heat transfer media, generating syngas, generating hydrogen, making electricity, and or the like.

The term “biodiesel,” as used herein, refers to components or streams suitable for direct use and/or blending into a diesel pool and/or a cetane supply derived from renewable sources. Suitable biodiesel molecules can include fatty acid esters, monoglycerides, diglycerides, triglycerides, lipids, fatty alcohols, alkanes, naphthas, distillate range materials, paraffinic materials, aromatic materials, aliphatic compounds (straight, branched, and/or cyclic), and/or the like. Biodiesel can be used in compression ignition engines, such as automotive diesel internal combustion engines, truck heavy duty diesel engines, and/or the like. In the alternative, the biodiesel can also be used in gas turbines, heaters, boilers, and/or the like. According to some embodiments, the biodiesel and/or biodiesel blends meet or comply with industrially accepted fuel standards, such as B20, B40, B60, B80, B99.9, B100, and/or the like.

The term “biodistillate” as used herein, refers to components or streams suitable for direct use and/or blending into aviation fuels (jet), lubricant base stocks, kerosene fuels, fuel oils, and/or the like. Biodistillate can be derived from renewable sources, and have any suitable boiling point range, such as a boiling point range of about 100 degrees Celsius to about 700 degrees Celsius, about 150 degrees Celsius to about 350 degrees Celsius, and/or the like. In certain embodiments, the biodistillate is produced from recently living plant or animal materials by a variety of processing technologies. According to one embodiment, the biodistillates can be used for fuel or power in a homogeneous charge compression ignition (HCCI) engine. HCCI engines may include a form of internal combustion with well-mixed fuel and oxidizer (typically air) compressed to the point of auto-ignition.

The term “consuming,” as used herein, refers to using up, utilizing, eating, devouring, transforming, and/or the like. According to some embodiments, consuming can include processes during and/or a part of cellular metabolism (catabolism and/or anabolism), cellular respiration (aerobic and/or anaerobic), cellular reproduction, cellular growth, fermentation, cell culturing, and/or the like.

The term “feedstock,” as used herein, refers to materials and/or substances used to supply, feed, provide for, and/or the like, such as to an organism, a machine, a process, a production plant, and/or the like. Feedstocks can include raw materials used for conversion, synthesis, and/or the like. According to some embodiments, the feedstock can include any material, compound, substance, and/or the like suitable for consumption by an organism, such as sugars, hexoses, pentoses, monosaccharides, disaccharides, trisaccharides, polyols (sugar alcohols), organic acids, starches, carbohydrates, and/or the like. According to some embodiments, the feedstock can include sucrose, glucose, fructose, xylose, glycerol, mannose, arabinose, lactose, galactose, maltose, other five carbon sugars, other six carbon sugars, other twelve carbon sugars, plant extracts containing sugars, other crude sugars, and/or the like. Feedstock can refer to one or more of the organic compounds listed above when present in the feedstock.

According to some embodiments, the feedstock can be fed into the fermentation using one or more feeds. In some embodiments, feedstock can be present in media charged to a vessel prior to inoculation. In some embodiments, feedstock can be added through one or more feed streams in addition to the media charged to the vessel.

According to some embodiments, the feedstock can include a lignocellulosic derived material, such as material derived at least in part from biomass and/or lignocellulosic sources.

According to some embodiments, the method and/or process can include addition of other materials and/or substances to aid and/or assist the organism, such as nutrients, vitamins, minerals, metals, water, and/or the like. The use of other additives are also within the scope of this disclosure, such as antifoam, flocculants, emulsifiers, demulsifiers, viscosity increases, viscosity reducers, surfactants, salts, other fluid modifying materials, and/or the like.

The term “organic,” as used herein, refers to carbon containing compounds, such as carbohydrates, sugars, ketones, aldehydes, alcohols, lignin, cellulose, hernicellulose, pectin, other carbon containing substances, and/or the like.

The term “biomass,” as used herein, refers to plant and/or animal materials and/or substances derived at least in part from living organisms and/or recently living organisms, such as plants and/or lignocellulosic sources. Non-limiting examples of materials comprising the biomass include proteins, lipids, and polysaccharides.

The term “cell culturing,” as used herein, refers to metabolism of carbohydrates whereby a final electron donor is oxygen, such as aerobically. Cell culturing processes can use any suitable organisms, such as bacteria, fungi (including yeast), algae, and/or the like. Suitable cell culturing processes can include naturally occurring organisms and/or genetically modified organisms.

The term “fermentation,” as used herein, refers both to cell culturing and to metabolism of carbohydrates where a final electron donor is not oxygen, such as anaerobically. Fermentation can include an enzyme controlled anaerobic breakdown of an energy rich compound, such as a carbohydrate to carbon dioxide and an alcohol, an organic acid, a lipid, and/or the like. In the alternative, fermentation refers to biologically controlled transformation of an inorganic or organic compound. Fermentation processes can use any suitable organisms, such as bacteria, fungi (including yeast), algae, and/or the like. Suitable fermentation processes can include naturally occurring organisms and/or genetically modified organisms.

Biological processes can include any suitable living system and/or item derived from a living system and/or a process. Biological processes can include fermentation, cell culturing, aerobic respiration, anaerobic respiration, catabolic reactions, anabolic reactions, biotransformation, saccharification, liquefaction, hydrolysis, depolymerization, polymerization, and/or the like.

The term “organism,” as used herein, refers to an at least relatively complex structure of interdependent and subordinate elements whose relations and/or properties can be largely determined by their function in the whole. The organism can include an individual designed to carry on the activities of life with organs separate in function but mutually dependent. Organisms can include a living being, such as capable of growth, reproduction, and/or the like.

The organism can include any suitable simple (mono) cell being, complex (multi) cell being, and/or the like. Organisms can include algae, fungi (including yeast), bacteria, and/or the like. The organism can include microorganisms, such as bacteria or protozoa. The organism can include one or More naturally occurring organisms, one or more genetically modified organisms, combinations of naturally occurring organisms and genetically modified organisms, and/or the like. Embodiments with combinations of multiple different organisms are within the scope of the disclosure. Any suitable combination or organism can be used, such as one or more organisms, at least about two organisms, at least about five organisms, about two organisms to about twenty organisms, and/or the like.

In one embodiment, the organism can be a single cell member of the fungal kingdom, such as a yeast, for example. Examples of oleaginous yeast that can be used include, but are not limited to the following oleaginous yeast: Candida apicola, Candida sp., Cryptococcus curvatus, Cryptococcus terricolus, Debaromyces hansenii, Endomycopsis vernalis, Geotrichum carabidarum, Geotrichum cucujoidarum, Geotrichum histendarum, Geotrichum silvicola, Geotrichum vulgare, Hyphopichia burtonii, Lipomyces lipoter, Lypomyces orentalis, Lipomyces starkeyi, Lipomyces tetrasporous, Pichia mexicana, Rodosporidium sphaerocarpum, Rhodosporidium toruloides Rhodotorula aurantiaca, Rhodotorula dairenensis, Rhodotorula diffluens, Rhodotorula glutinus, Rhodotorula glutinis, Rhodotorula gracilis, Rhodotorula graminis, Rhodotorula minute, Rhodotorula mucilaginosa, Rhodotorula mucilaginosa, Rhodotorula terpenoidalis, Rhodotorula toruloides, Sporobolomyces alborubescens, Starmerella bombicoia, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri, Trichosporon montevideense, Trichosporon pullulans, Trichosporon sp., Wickerhamomyces canadensis, Yarrowia lipolytica, and Zygoascus meyerae.

The organism can operate, function, and/or live under any suitable conditions, such as anaerobically, aerobically, photosynthetically, heterotrophically, and/or the like.

The term “oleaginous,” as used herein, refers to oil bearing, oil containing and/or producing oils, lipids, fats, and/or other oil-like substances. The oil, lipid, fat, and/or other oil-like substances may be produced inside or outside the cell. Oleaginous may include organisms that produce at least about 20 percent by weight of oils, at least about 30 percent by weight of oils, at least about 40 percent by weight oils, at least about 50 percent by weight oils, at least about 60 percent by weight oils, at least about 70 percent by weight oils, at least about 80 percent by weight oils, and/or the like. Oleaginous may refer to a microorganism during culturing, lipid accumulation, at harvest conditions, and/or the like.

The term “genetic engineering,” as used herein, refers to intentional manipulation and/or modification of at least a portion of a genetic code and/or expression of a genetic code of an organism.

The term “genetic modification,” as used herein, refers to any method of introducing a genetic change to an organism. Non-limiting examples include genomic mutagenesis, addition and/or removal of one or more genes, portions of proteins, promoter regions, noncoding regions, chromosomes, and/or the like. Genetic modification can be random or non-random. Genetic modification can comprise, for example, mutations, and can be insertions, deletions, point mutations, substitutions, and any other mutation. Genetic modification can also be used to refer to a genetic difference a non-wild type organism and a wild type organism.

The terms “unmodified organism” or “unmodified microorganism,” as used herein, refer to organisms, cultures, single cells, biota, and/or the like at least generally without intervening actions by exterior forces, such as humankind, machine, and/or the like. As used herein, an unmodified microorganism is typically the particular microorganism as it exists prior to introduction of a genetic modification according to the application. In most embodiments, an unmodified microorganism is the wild type strain of the microorganism. However, the unmodified microorganism as defined herein can be an organism that was genetically altered prior to the introduction of the genetic modification according to this disclosure. For example, a yeast strain available from ATCC that comprises a knockout mutation of a certain gene would be considered an unmodified microorganism according to this definition. The term unmodified microorganism also encompasses organisms that do not have a genetic modification associated with production of polysaccharides or fermentation broth viscosity.

In some embodiments, producing an organism includes where the organism includes fatty acids and/or results in an organism containing fatty acids, such as within or on one or more vesicles and/or pockets. In the alternative, the fatty acid can be relatively uncontained within the cell and/or outside the cell, such as relatively free from constraining membranes. Producing the organism can include cellular reproduction (more cells) as well as cell growth (increasing a size and/or contents of the cell, such as by increasing a fatty acid content). Reproduction and growth can occur at least substantially simultaneously with each other, at least substantially exclusively of each other, at least partially simultaneously and at least partially exclusively, and/or the like.

Polysaccharides (also called “glycans”) are carbohydrates made up of monosaccharides joined together by glycosidic linkages. Polysaccharides are broadly defined molecules, and the definition includes intercellular polysaccharides, secreted polysaccharides, exocellular polysaccharides, cell wall polysaccharides, and the like. Cellulose is an example of a polysaccharide that makes up certain plant cell walls. Cellulose can be depolymerized by enzymes to produce monosaccharides such as xylose and glucose, as well as larger disaccharides and oligosaccharides. The quantity of each monosaccharides component following depolyrnerization of polysaccharides is defined herein as a monosaccharide profile. Certain polysaccharides comprise non-carbohydrate substituents, such as acetate, pyruvate, succinate, and phosphate.

The term “fatty acids,” as used herein, refer to saturated and/or unsaturated monocarboxylic acids, such as in the form of glycerides in fats and fatty oils. Glycerides can include acylglycerides, monoglycerides, diglycerides, triglycerides, and/or the like. Fatty acid also refers to carboxylic acids having straight or branched hydrocarbon groups having from about 8 to about 30 carbon atoms. The hydrocarbon groups including from 1 to about 4 sites of unsaturation, generally double or pi bonds. Examples of such fatty acids are lauric acid, steric acid, palmitic acid, oleic acid, linoleic acid, linolenic acid, arachidonic acid, elaidic acid, linoelaidicic acid, eicosenoic acid, phytanic acid, behenic acid, and adrenic acid.

Double bonds refer two pairs of electrons shared by two atoms in a molecule.

The term “unit,” as used herein, refers to a single quantity regarded as a whole, a piece and/or complex of apparatus serving to perform one or more particular functions and/or outcomes, and/or the like.

The term “stream,” as used herein, refers to a flow and/or a supply of a substance and/or a material, such as a steady succession. Flow of streams can be continuous, discrete, intermittent, batch, semibatch, semicontinuous, and/or the like.

The term “vessel,” as used herein, refers to a container and/or holder of a substance, such as a liquid, a gas, a fermentation broth, and/or the like. Vessels can include any suitable size and/or shape, such as at least about 1 liter, at least about 1,000 liters, at least about 100,000 liters, at least about 1,000,000 liters, at least about 1,000,000,000 liters, less than about 1,000,000 liters, about 1 liter to about 1,000,000,000 liters, and/or the like. Vessels can include tanks, reactors, columns, vats, barrels, basins, and/or the like. Vessels can include any suitable auxiliary equipment, such as pumps, agitators, aeration equipment, heat exchangers, coils, jackets, pressurization systems (positive pressure and/or vacuum), control systems, and/or the like.

The term “dispose,” as used herein, refers to put in place, to put in location, to set in readiness, and/or the like. The organism can be freely incorporated into a fermentation broth (suspended), and/or fixed upon a suitable media and/or surface within at least a portion of the vessel. The organism can be generally denser than the broth (sink), generally lighter than the broth (float), generally neutrally buoyant with respect to the broth, and/or the like.

The term “adapted,” as used herein, refers to make fit for a specific use, purpose, and/or the like.

The term “meeting,” as used herein, refers to reaching, obtaining, satisfying, equaling, and/or the like.

The term “exceeding,” as used herein, refers to extending beyond, to surpassing, and/or the like. According to some embodiments, exceeding includes at least 2 percent above threshold amount and/or quantity.

Cell density (of the organism) measured in grams dry weight per liter (of the fermentation media or broth), measures and/or indicates productivity of the organism, utilization of the fermentation media (broth), and/or utilization of fermentation vessel volume. Increased cell density can result in increased production of a particular product and increased utilization of equipment (lower capital costs). Generally, increased cell density is beneficial, but too high a cell density can result in higher mixing and pumping costs (increased viscosity) and/or difficulties in removing heat (lower heat transfer coefficient), and/or the like.

The term “viscosity,” as used herein, refers to the physical property of fluids that determines the internal resistance to shear forces. Viscosity can be measured by several methods, including for example a viscometer, with typical units of centipoise (cP). Viscosity can also be measured using other known devices, such as a rheometer.

The term “mass transfer,” as used herein, refers to the net movement of mass from one location to another. Often, chemical species transfer between two phases through an interface or diffusion through a phase. The driving force for mass transfer is a difference in concentration; the random motion of molecules causes a net transfer of mass from an area of high concentration to an area of low concentration. For separation processes, thermodynamics determines the extent of separation, while mass transfer determines the rate at which the separation will occur. One important mass transfer is that of oxygen and other nutrients into the fermentation broth.

The amount of mass transfer rate can be quantified through the calculation and application of mass transfer coefficients, (m/s) which is a diffusion rate constant that relates the mass transfer rate, mass transfer area, and concentration gradient as driving force. This can be used to quantify the mass transfer between phases, immiscible and partially miscible fluid mixtures (or between a fluid and a porous solid). Quantifying mass transfer allows for design and manufacture of fermentation process equipment that can meet specified requirements, estimate what will happen in real life situations.

The term “density,” as used herein, refers to a mass per unit volume of a material and/or a substance. Cell density refers to a mass of cells per unit volume, such as the weight of living cells per unit volume. It is commonly expressed as grams of dry cells per liter. The cell density can be measured at any suitable point in the method, such as upon commencing fermentation, during fermentation, upon completion of fermentation, over the entire batch, and/or the like.

The term “FAME,” as used herein, refers to a fatty acid methyl ester. The term FAME may also be used to describe the assay used to determine the fatty acid methyl ester quantity or percentage in a microorganism.

The term “free fatty acid equivalent,” as used herein, means FAME determined using test method Celb-89 from the American Oil Chemists Society, and multiplied by a factor of 0.953.

The term “yield,” as used herein, refers to an amount and/or quantity produced and/or returned as compared to a quantity consumed. As non-limiting examples, the quantity consumed can be sugars, carbon, oxygen, or any other nutrient. “Yield” can also refer to an amount and/or quantity produced and/or returned as compared to a time period elapsed.

the terms “fermentation yield,” “fatty acid yield,” or “sugar yield,” as used herein, mean the total estimated free fatty acid equivalent produced (by weight) divided by the total sugar consumed during the fermentation process. (by weight).

The fatty acid yield can be measured at any suitable point in the method, such as upon commencing fermentation, during fermentation, upon completion of fermentation, over the entire batch, and/or the like.

Generally, a higher fatty acid content is desired and can provide for easier extraction and/or removal of the fatty acids from a remainder and/or residue of cellular material, as well as increased utilization and/or productivity for the feedstock and/or equipment.

Generally, a higher fatty acid productivity results in a more economic process since making product more rapidly (i.e., reduced cycle times) is desired.

A higher fatty acid yield is generally preferred as it indicates carbon conversion from the sugar into fatty acid and not byproducts and/or cell mass.

Fatty acid yield on oxygen expressed as grams of fatty acids produced per gram of oxygen consumed basis measures and/or indicates an amount and/or rate of oxygen used to produce the fatty acids. A higher oxygen demand can increase capital expenses and/or operating expenses.

The term “content,” as used herein, refers to an amount of specified material contained. Dry mass basis refers to being at least substantially free from water. The fatty acid content can be measured at any suitable point in the method, such as upon commencing fermentation, during fermentation, upon completion of fermentation, over the entire batch, and/or the like.

The term “productivity,” as used herein, refers to a quality and/or state of producing and/or making, such as a rate per unit of volume. The fatty acid productivity can be measured at any suitable point in the method, such as upon commencing fermentation, during fermentation, upon completion of fermentation, over the entire batch, and/or the like. The productivity can be measured on a fixed time, such as noon to noon each day. In the alternative, the productivity can be measured on a suitable rolling basis, such as for any 24 period. Other bases for measuring productivity are within the scope of the disclosure.

### 2. MICROORGANISMS

In one aspect, disclosed is an oleaginous microorganism suitable for production of renewable materials.

Some microorganisms produce significant quantities of non-lipid metabolites, such as, for example, polysaccharides. Polysaccharide biosynthesis is known to use a significant proportion of the total metabolic energy available to cells. As disclosed herein, mutagenesis of lipid-producing cells followed by screening for reduced or eliminated polysaccharide production generates novel strains that are capable of producing higher yields of lipids. These significant and unexpected improvements may result from an improved mass transfer characteristic of the culture, a higher flux of carbon to fatty acids, or both of these mechanisms. For some microorganisms, the increase lipid yield may be through a mechanism that is not yet characterized.

In certain embodiments, the microorganisms disclosed comprise a modification. In some embodiments, the modification is a genetic modification not present in an unmodified microorganism.

The genetic modification can be introduced by many methods. In certain embodiments, the genetic modification is introduced by genetic engineering. In other embodiments, the genetic modification is introduced by random mutagenesis.

In particular embodiments, the modification affects polysaccharide synthesis. In other embodiments, the modification affects one or more genes encoding a protein that contributes to polysaccharide synthesis. In other embodiments, the modification affects one or more regulatory genes that encode proteins that control polysaccharide synthesis. In still other embodiments, the modification affects one or more non-coding regulatory regions. In other embodiments, one or more genes is up-regulated or down-regulated such that polysaccharide production is decreased.

In still other embodiments, the modification affects polysaccharide transport and/or secretion. In some embodiments, the modification affects one or more genes encoding a protein that contributes to polysaccharide transport and/or secretion. In other embodiments, the modification affects one or more regulatory genes that encode proteins that control polysaccharide transport and/or secretion. In still other embodiments, the modification affects one or more non-coding regulatory regions. In other embodiments, one or more genes is up-regulated or down-regulated such that polysaccharide transport and/or secretion is decreased.

In other embodiments, the genetic modification affects one or more genes that control fatty acid synthesis. These genes include branch points in the metabolic pathway of fatty acids. In other embodiments, the gene is up-regulated or down-regulated such that lipid production is increased. Examples of enzymes suitable for up-regulation according to the disclosed methods include pyruvate dehydrogenase, which plays a role in converting pyruvate to acetyl-CoA. Up-regulation of pyruvate dehydrogenase can increase production of acetyl-CoA, and thereby increase fatty acid synthesis. Acetyl-CoA carboxylase catalyzes the initial step in fatty acid synthesis. Accordingly, this enzyme can be up-regulated to increase production of fatty acids. Fatty acid production can also be increased by up-regulation of acyl carrier protein (ACP), which carries the growing acyl chains during fatty acid synthesis. Glycerol-3-phosphate acyltransferase catalyzes the rate-limiting step of fatty acid synthesis. Up-regulation of this enzyme can increase fatty acid production.

Examples of enzymes potentially suitable for down-regulation according to the disclosed methods include citrate synthase, which consumes acetyl-CoA as part of the tricarboxylic acid (TCA) cycle. Down-regulation of citrate synthase can force more acetyl-CoA into the fatty acid synthetic pathway.

Any species of organism that produces suitable lipid or hydrocarbon can be used, although microorganisms that naturally produce high levels of suitable lipid or hydrocarbon are preferred. Production of hydrocarbons by microorganisms is reviewed by Metzger et al. Appl Microbial Biotechnol (2005) 66: 486-496 and A Look Back at the U.S. Department of Energy's Aquatic Species Program: Biodiesel from Algae, NREUTP-580-24190, John Sheehan, Terri Dunahay, John Benemann and Paul Roessler (1998).

In some embodiments, a microorganism producing a lipid or a microorganism from which a lipid can be extracted, recovered, or obtained, is a fungus. Examples of fungi that can be used include, but are not limited to the following genera and species of fungi: Mortierella, Mortierrla vinacea, Mortierella alpine, Pythium debaryanum, Mucor circinelloides, Aspergillus ochraceus, Aspergillus terreus, Pennicillium iilacinum, Hensenulo, Chaetomium, Cladosporium, Malbranchea, Rhizopus, and Pythium.

In a certain embodiment, the disclosed oleaginous modified microorganism is a yeast. Examples of gene mutation in oleaginous yeast can be found in the literature (see Bordes et al, J. Microbial. Methods, June 27 (2007)). In certain embodiments, the yeast belongs to the genus Rhodotorula, Pseudozyma, or Sporidiobolus. Examples of oleaginous yeast that can be used include, but are not limited to the following oleaginous yeast: Candida apicola, Candida sp., Cryptococcus curvatus, Cryptococcus terricolus, Debaromyces hansenii, Endomycopsis vernalis, Geotrichum carabidarum, Geotrichum cucujoidarum, Geotrichum histendarum, Geotrichum silvicola, Geotrichum vulgare, Hyphopichia burtonii, Lipomyces lipofer, Lypomyces orentalis, Lipomyces starkeyi, Lipomyces tetrasporous, Pichia mexicana, Rodosporidium sphaerocarpum, Rhodosporidium toruloides Rhodotorula aurantiaca, Rhodotorula dairenensis, Rhodotorula diffluens, Rhodotorula glutinus, Rhodotorula glutinis, Rhodotorula gracilis, Rhodotorula graminis, Rhodotorula minute, Rhodotorula mucilaginosa, Rhodotorula mucilaginosa, Rhodotorula terpenoidalis, Rhodotorula toruloides, Sporobolomyces alborubescens, Starmerella bombicola, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri, Trichosporon montevideense, Trichosporon pullulans, Trichosporon sp., Wickerhamomyces canadensis, Yarrowia lipolytica, and Zygoascus meyerae.

In other embodiments, the yeast belongs to the genus Sporidiobolus pararoseus. In a specific embodiment, the disclosed microorganism is the microorganism corresponding to ATCC Deposit No. PTA-12508 (Strain MK29404 (Dry1-13J)). In another specific embodiment, the microorganism is the microorganism corresponding to ATCC Deposit No. PTA-12509 (Strain MK29404 (Dry1-182J)), In another specific embodiment, the microorganism is the microorganism corresponding to ATCC Deposit No. PTA-12510 (Strain MK29404 (Dry1-173N)). In another specific embodiment, the microorganism is the microorganism corresponding to ATCC Deposit No. PTA-12511 (Strain MK29404 (Dry55)). In another specific embodiment, the microorganism is the microorganism corresponding to ATCC Deposit No. PTA-12512 (Strain MK29404 (Dry41)). In another specific embodiment, the microorganism is the microorganism corresponding to ATCC Deposit No. PTA-12513 (Strain MK29404 (Dry1)). In another specific embodiment, the microorganism is the microorganism corresponding to ATCC Deposit No. PTA-12515 (Strain MK29404 (Dry1-147D)). In another specific embodiment, the microorganism is the microorganism corresponding to ATCC Deposit No, PTA-12516 (Strain MK29404 (Dry1-72D)).

In other embodiments, the yeast belongs to the genus Rhodotorula ingeniosa. In a specific embodiment, the disclosed microorganism is the microorganism corresponding to ATCC Deposit No. PTA-12506 (Strain MK29794 (KDry16-1)). In another specific embodiment, the disclosed microorganism is the microorganism corresponding to ATCC Deposit No. PTA-12507 (Strain MK29794 (KDry7)). In another specific embodiment, the disclosed microorganism is the microorganism corresponding to ATCC Deposit No. PTA-12514 (Strain MK29794 (K200 Dry1)). In another specific embodiment, the disclosed microorganism is the microorganism corresponding to ATCC Deposit No. PTA-12517 (Strain MK29794 (33 Dry1)).

In certain embodiments, the modified yeast comprises a dry morphology, while the unmodified yeast does not comprise a dry morphology. In other embodiments, the unmodified yeast comprises the morphology of the wild type yeast strain.

### 3. CULTURE CONDITIONS

According to certain embodiments, the oleaginous microorganism is grown in culture, such as for example during manufacture In some embodiments, the culture of the modified microorganism comprises substantially similar conditions as the culture of the unmodified microorganism.

Microorganisms can be cultured both for purposes of conducting genetic manipulations and for subsequent production of hydrocarbons (e.g., lipids, fatty acids, aldehydes, alcohols, and alkanes). The former type of culture is conducted on a small scale and initially, at least, under conditions in which the starting microorganism can grow. For example, if the starting microorganism is a photoautotroph the initial culture is conducted in the presence of light. The culture conditions can be changed if the microorganism is evolved or engineered to grow independently of light. Culture for purposes of hydrocarbon production is usually conducted on a large scale. In certain embodiments, during culture conditions a fixed carbon source is present. The culture can also be exposed to light at various times during culture, including for example none, some, or all of the time.

For organisms able to grow on a fixed carbon source, the fixed carbon source can be, for example, glucose, fructose, sucrose, galactose, xylose, mannose, rhamnose, N-acetylglucosamine, glycerol, floridoside, and/or glucuronic acid. The one or more carbon source(s) can be supplied at a concentration of at least about 50 μM, at least about 100 μM, at least about 500 μM, at least about 5 mM, at least about 50 mM, and at least about 500 mM, of one or more exogenously provided fixed carbon source(s). Some microorganisms can grow by utilizing a fixed carbon source such as glucose or acetate in the absence of light. Such growth is known as heterotrophic growth.

Other culture parameters can also be manipulated. Non-limiting examples include manipulating the pH of the culture media, the identity and concentration of trace elements, and other media constituents. Culture media may be aqueous, such as containing a substantial portion of water.

Modifying the conditions of fermentation is one way to attempt to increase yield of desired lipid or other biological product. However, this strategy has limited value, for the conditions that promote lipid production (high carbon to nitrogen ratios) also promote polysaccharide production.

Process conditions can be adjusted to decrease the yield of polysaccharides to reduce production cost. For example, in certain embodiments, a microorganism is cultured in the presence of a limiting concentration of one or more nutrients, such as, for example, carbon and/or nitrogen, phosphorous, or sulfur, while providing an excess of fixed carbon energy such as glucose. Nitrogen limitation tends to increase microbial lipid yield over microbial lipid yield in a culture in which nitrogen is provided in excess. The microorganism can be cultured in the presence of a limiting amount of a nutrient for a portion of the total culture period or for the entire period. In particular embodiments, the nutrient concentration is cycled between a limiting concentration and a non-limiting concentration at least twice during the total culture period.

To increase lipid yield, acetic acid can be employed in the feedstock for an oleaginous microorganism. Acetic acid feeds directly into the point of metabolism that initiates fatty acid synthesis (i.e., acetyl-CoA); thus providing acetic acid in the culture can increase fatty acid production, Generally, the microorganism is cultured in the presence of a sufficient amount of acetic acid to increase microbial lipid yield, and/or microbial fatty acid yield, specifically, over microbial lipid (e.g., fatty acid) yield in the absence of acetic acid.

In another embodiment, lipid yield is increased by culturing a microorganism in the presence of one or more cofactor(s) for a lipid pathway enzyme (e.g., a fatty acid synthetic enzyme). Generally, the concentration of the cofactor(s) is sufficient to increase lipid (e.g., fatty acid) yield over microbial lipid yield in the absence of the cofactor(s). In a particular embodiment, the cofactor(s) are provided to the culture by including in the culture a microorganism containing an exogenous gene encoding the cofactor(s). Alternatively, cofactor(s) may be provided to a culture by including a microorganism containing an exogenous gene that encodes a protein that participates in the synthesis of the cofactor. In certain embodiments, suitable cofactors include any vitamin required by a lipid pathway enzyme, such as, for example: biotin, pantothenate. In other embodiments, genes encoding cofactors or that participate in the synthesis of such cofactors can be introduced into microorganisms (e.g., microalgae, yeast, and others).

### 4. POLYSACCHARIDES

In another aspect, the oleaginous microorganisms disclosed in the application produce a polysaccharide. In some embodiments, the modified microorganism produces a polysaccharide. In other embodiments, the unmodified microorganism produces a polysaccharide. In still other embodiments, both the modified microorganism and the unmodified microorganism produce a polysaccharide.

Polysaccharides, when synthesized, may be retained within the cell (intracellular), disposed within the cell wall, and/or secreted outside the cell (exocellular). Microorganisms that have low levels of exocellular polysaccharide can be identified based on visual observation of colony morphology on agar plates. Colonies that produce higher levels of exocellular polysaccharide are wet in appearance and very soft. If the plate is inverted (placed upside down) the colony will drip onto the other side of the plate. This morphology is characteristic of cells that produce large amounts of exocellular polysaccharide. Low level exocellular polysaccharide mutants can be identified by a colony morphology that is and not visibly wet, expressed herein as a “dry” morphology. These low polysaccharide colonies are not soft but stiff and powdery.

In one embodiment, the modified microorganism comprises a dry morphology. In some embodiments, the modified microorganism comprises a dry morphology, while the unmodified microorganism does not comprise a dry morphology. In certain embodiments, the unmodified microorganism comprises the morphology of the wild type microorganism.

One well characterized exocellular polysaccharide is the xanthan polysaccharide. (Shu and Yang, Biotechnol Bioeng. March 5; 35(5):454-68 (1990)), In the xanthan pathway, most research efforts have sought to increase the production of the xanthan polysaccharide for industrial applications. However, several problems with fermentation are observed when the levels of secreted polysaccharide rise, and the fermentation becomes more costly.

Disclosed herein are novel microorganisms that produce a polysaccharide at reduced levels. Polysaccharide production of the disclosed microorganisms may be reduced at any level, including at the gene, protein, protein folding or modification, synthesis pathway, or cellular/extracellular level. The invention is not limited to any specific mechanism of polysaccharide reduction.

In certain embodiments where both modified and unmodified microorganism produce exocellular polysaccharide, the modified microorganism produces less exocellular polysaccharide than the unmodified microorganism. In certain embodiments, the unmodified microorganism typically comprises the wild type strain of the microorganism. In certain embodiments, the microorganism produces at least 4 times less polysaccharide than the unmodified microorganism. In other embodiments, the microorganism produces at least 1.5, 2.0, 2.5, 3.0, 3.5, 5.0, 6.0, 7.0, 8.0, 9.0, 10, 15, 20, 30, 40 or 50 times less exocellular polysaccharide than the unmodified microorganism. In some embodiments, the production of polysaccharide is lower in the modified microorganism because of a mutation in a gene associated with polysaccharides.

Exocellular polysaccharides may be found in the fermentation broth produced or resulting from by the disclosed microorganisms. Fermentation broth may include, among others, a carbon source, nutrients, organism bodies, organism secretions, water, byproducts, waste products, and/or the like. Exocellular polysaccharides are typically found outside of the cell due to cellular export (e.g. secretion) or by disruption of a cell membrane, such as during cell death. The polysaccharide is also generally known or referred to as an “exopolysaccharide” if found outside of the cell. The modified microorganism, the unmodified microorganism, or both may produce a fermentation broth comprising a polysaccharide. Also disclosed herein is an unmodified microorganism that produces a polysaccharide, but the modified microorganism does not produce a polysaccharide.

Exocellular polysaccharides can be quantified using several different metrics as can readily be calculated by one of ordinary skill in the art. In one embodiment, the exocellular polysaccharide is quantified as mass of the polysaccharide per unit volume of the fermentation broth produced by the microorganism. The mass of the polysaccharide per unit volume of the fermentation broth produced by the microorganisms according to the disclosure can be readily calculated by one of ordinary skill in the art. Other non-limiting metrics that can be used to quantify exocellular polysaccharide include: absolute level (grams/volume) of total soluble polysaccharide; absolute level of individual sugars (grams/volume) of total hydrolyzed soluble polysaccharide; ratios of soluble polysaccharide to total biomass; ratio of soluble biomass to lean biomass; ratio of soluble polysaccharide to lipid; ratio of polysaccharide to extractable lipid; quantity of polysaccharide per cell; absolute level of viscosity; and/or ratio of viscosity to soluble polysaccharide using any of the above values. Determination of these metrics is well within ordinary skill in the art.

In certain embodiments, the modified microorganism produces less than about 22.8 grams of exocellular polysaccharide per liter of fermentation broth. In other embodiments, the modified microorganism produces less than about 6 grams per liter of fermentation broth. In other embodiments, the modified microorganism produces less than about 3 grams exocellular polysaccharide per liter of fermentation broth. In other embodiments, the modified microorganism produces less than about 1 gram per liter of fermentation broth. In other embodiments, the microorganism produces less than about 0.5, 0.25, 0.1, 0.05, 0.01, or less exocellular polysaccharide per liter of fermentation broth.

In certain embodiments where both modified and unmodified microorganisms produce a fermentation broth comprising an exocellular polysaccharide, the modified microorganism produces a fermentation broth comprising less polysaccharide than an equal volume of fermentation broth of the unmodified microorganism. In one embodiment, the modified microorganism produces at least about 2 times less exocellular polysaccharide per liter of fermentation broth than the unmodified microorganism. In other embodiments, the modified microorganism produces at least about 4 times less polysaccharide per liter of fermentation broth than the unmodified microorganism. In other embodiments, the modified microorganism produces at least 5, 6, 7, 8, 9, 10, 15, 20, 30, 40, 50, 100, or 1000 times less exocellular polysaccharide per liter of fermentation broth than the unmodified microorganism. In other embodiments, the modified microorganism produces at least 2, 5, 10, 20, 30, 40, 50, 75, 90, or 99 percent less polysaccharide per liter of fermentation broth than the unmodified microorganism.

Exocellular polysaccharides can also be quantified by calculating the ratio of lipid to polysaccharide in the fermentation broth produced by the described microorganisms. This calculation can be readily obtained by one of ordinary skill in the art.

In certain embodiments, the modified microorganisms according to the invention produce a fermentation broth comprising a lipid to exocellular polysaccharide ratio of greater than about 2. In other embodiments, the modified microorganisms produce a fermentation broth comprising a lipid to exocellular polysaccharide ratio of about 10. In still other embodiments, the modified microorganisms produce a fermentation broth comprising a lipid to exocellular polysaccharide ratio of greater than about 10. In further embodiments, the modified microorganisms produce a fermentation broth comprising a lipid to exocellular polysaccharide ratio of about 50. In further embodiments, the modified microorganisms produce a fermentation broth comprising a lipid to exocellular polysaccharide ratio of about 70. In further embodiments, the modified microorganisms produce a fermentation broth comprising a lipid to exocellular polysaccharide ratio of about 100, 200, 300, 400, 500, or 1000 or greater.

Exocellular polysaccharides can be quantified by calculating the mass of the polysaccharide per total biomass of the fermentation broth produced by the disclosed microorganisms. This calculation can be readily obtained by one of ordinary skill in the art.

In certain embodiments, the modified microorganisms produce a fermentation broth comprising about 0.20 grams exocellular polysaccharide per gram of total broth biomass (see Table 4). In other embodiments, the modified microorganisms produce a fermentation broth comprising at least about 0.04 grams of polysaccharide per gram of total broth biomass. In further embodiments, the modified microorganisms produce a fermentation broth comprising about 0.1, 0.5, 1.0, or 10.0 grams exocellular polysaccharide per 100 grams of total broth biomass.

In certain embodiments where both modified and unmodified microorganisms produce a fermentation broth comprising an exocellular polysaccharide, the modified microorganism produces a fermentation broth comprising less grams of exocellular polysaccharide per gram of total broth biomass than the fermentation broth of the unmodified microorganism (see Table 4). In other embodiments, the modified microorganism produces a fermentation broth comprising about 2 times less grams of exocellular polysaccharide per gram of total broth biomass than the fermentation broth of the unmodified microorganism. In yet other embodiments, the modified microorganism produces a fermentation broth comprising about 5 times less grams of exocellular polysaccharide per gram of total broth biomass than the fermentation broth of the unmodified microorganism. In other embodiments, the modified microorganism produces at least 5, 6, 7, 8, 9, 10, 15, 20, 30, 40, 50, 100, or 1000 times less exocellular polysaccharide per gram of total broth biomass than the fermentation broth of the unmodified microorganism. In other embodiments, the modified microorganism produces at least 2, 5, 10, 20, 30, 40, 50, 75, 90, or 99 percent less exocellular polysaccharide per gram of total broth biomass than the fermentation broth of the unmodified microorganism.

The novel modified microorganisms described herein produce a specific fermentation broth. This fermentation broth comprises certain biological components in specific ratios. In a certain embodiment, the fermentation broth produced by the modified microorganism comprises a lipid to exocellular polysaccharide ratio of greater than about 2. In another embodiment, the fermentation broth produced by the modified microorganism comprises a lipid to exocellular polysaccharide ratio of about 10. In another embodiment, the fermentation broth produced by the modified microorganism comprises a lipid to exocellular polysaccharide ratio of greater than about 10. In further embodiments, the fermentation broth produced by the modified microorganism comprises a lipid to exocellular polysaccharide ratio of about 100, 200, 300, 400, 500, or 1000 or greater.

Polysaccharide structure generally comprises monosaccharides joined together by glycosidic linkages. Both unmodified and modified microorganisms produce a polysaccharide in many of the described embodiments. As disclosed herein are novel modified microorganisms that produce a polysaccharide at reduced levels than the unmodified microorganisms. In these embodiments where both modified and unmodified microorganism produce an exocellular polysaccharide, the polysaccharide may have the same structure for both microorganisms, and the modified microorganism may produce less quantity of the same polysaccharide structure as the unmodified microorganism. Also contemplated, however, are modified microorganisms that produce a different exocellular polysaccharide structure than the unmodified microorganism. In these embodiments, the novel modified microorganisms produce an exocellular polysaccharide at reduced levels because the polysaccharide structure is different than the unmodified microorganism. For example, a modified microorganism may produce an exocellular polysaccharide with a lower molecular weight than the unmodified microorganism, leading to a reduced polysaccharide mass per volume of fermentation broth.

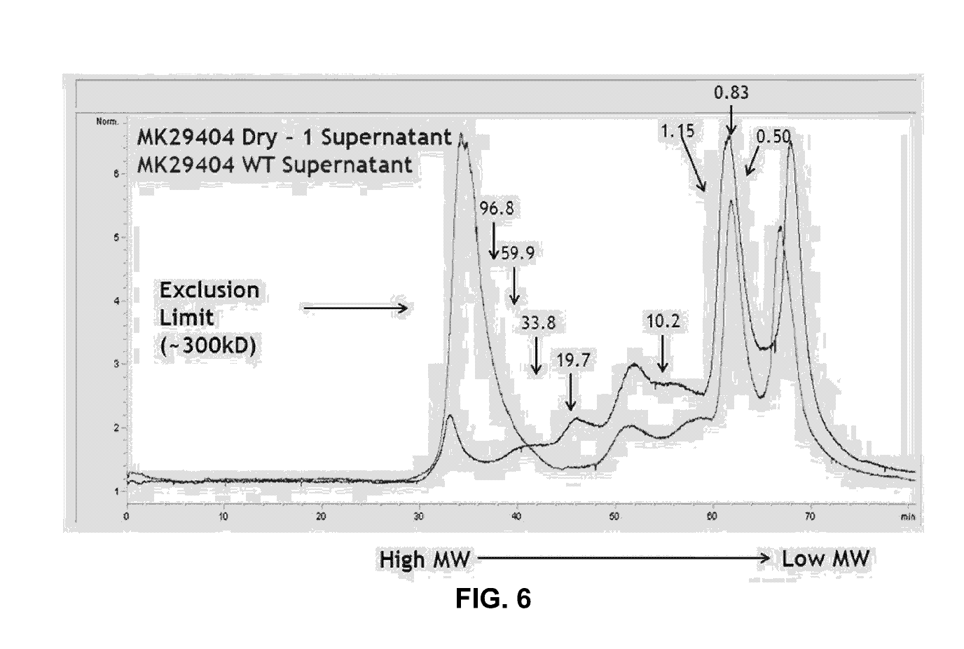

The modified microorganisms as disclosed in certain embodiments produce a different exocellular polysaccharide than the unmodified microorganism. The structure of the exocellular polysaccharide produced by the modified microorganism is altered as compared to the unmodified organism. In many of these particular embodiments, the exocellular polysaccharide produced by the modified microorganism has a different molecular weight than the polysaccharide produced by the unmodified microorganism. In one embodiment, the modified microorganism produces a exocellular polysaccharide with a lower molecular weight than the exocellular polysaccharide produced by the unmodified microorganism. (see FIG. 6)

Polysaccharide structure can be analyzed through several methods, including for example: HPLC, size exclusion chromatography (SEC), ion exchange chromatography (IEC), sedimentation analysis, gradient centrifugation, and ultra-filtration (see for example Prosky L, et al., J. Assoc. Off. Analytical Chem. 71:1017-1023 (1988); Deniaud, et al., Int. J. Biol. Macromol., 33:9-18 (2003). These methods can involve size fractionation of microorganism extracts. SEC techniques and ultrafiltration methods are often employed. The basic principles of SEC are further described in, for example, Hoagland, et al., J. Agricultural Food Chem., 41(8):1274-1281(1993). The appropriate columns for fractionating particular ranges can be readily selected and effectively used to resolve the fractions, e.g. Sephacryl S 100 HR, Sephacryl S 200 HR, Sephacryl S 300 HR, Sephacryl S 400 HR and Sephacryl S 500 HR or their equivalents. In an analogous fashion, Sepharose media or their equivalents, e.g. Sepharose 6B, 4B, 2B, can be used.

Purification of the polysaccharides or polysaccharide complexes with protein could be achieved in combination with other chromatography techniques, including affinity chromatography, IEC, hydrophobic interaction chromatography, or others.

Ultrafiltration of the samples could be performed using molecular membranes with appropriate molecular mass cutoffs. The specific membranes and procedures used to effect fractionation are widely available to those skilled in the art.

Polysaccharides can also be detected using gel electrophoresis (see for example Goubet, et al., Anal Biochem. 321:174-82 (2003); Goubet, et al., Anal Biochem. 300:53-68 (2002), Other assays can be used to detect particular polysaccharides as needed, such as the phenol: sulfuric acid assay for detecting carbohydrates (see Cuesta G., et al., J Microbiol Methods. 2003 January; 52(1):69-73); and Braz et al, J. Med. Biol. Res. 32(5):545-50 (1999); Panin et al., Olin. Chem. November; 32:2073-6 (1986)).

The different exopolysaccharide compositions, structures and/or productivities may be a direct or indirect result of the genetic modification of the modified microorganism. The change can be due to any biological process, and is not limited to any biological mechanism or pathway. The change may affect the genetics of the microorganism, or transcription, translation, post-translational modification, protein folding, monosaccharide assembly, or any other biological process involved in the synthesis of the polysaccharide. In some embodiments, mechanism for producing the polysaccharide may be unknown. In other embodiments, the polysaccharide produced by the modified microorganism may be a previously uncharacterized polysaccharide.

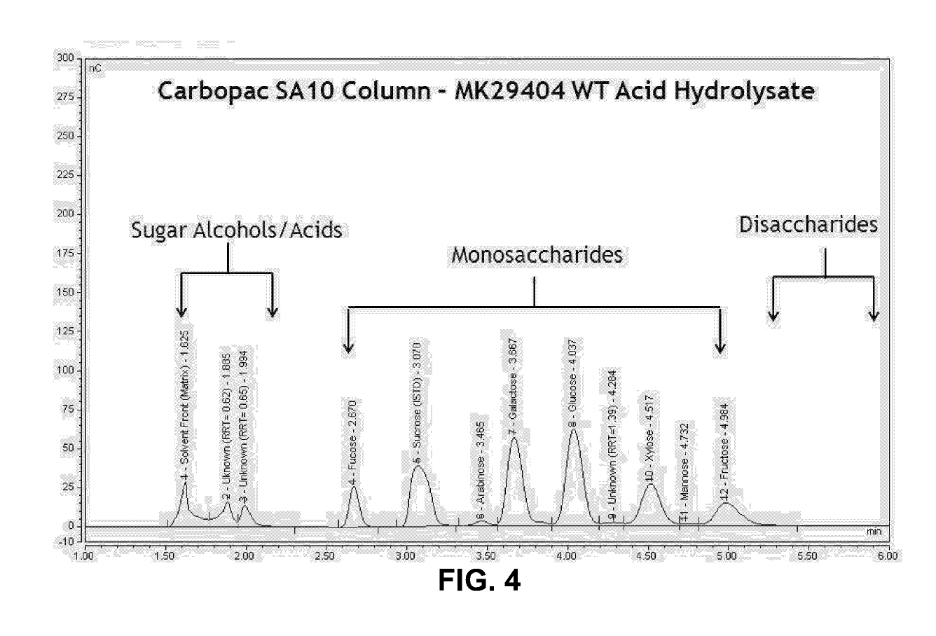

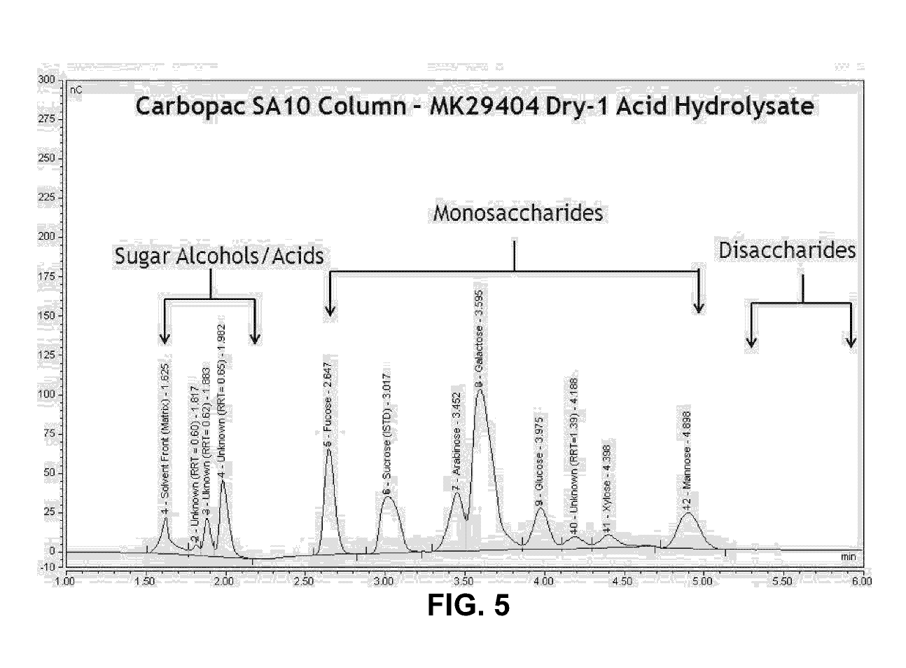

In another aspect, the modified microorganisms as disclosed produce an exocellular polysaccharide comprising different monosaccharide components than the monosaccharide components of the polysaccharide produced by the unmodified microorganism (compare FIG. 4 and FIG. 5) According to some embodiments, the modified microorganism produces an exocellular polysaccharide comprising a different monosaccharide profile than the polysaccharide produced by the unmodified microorganism (compare Table 5 and 6).

Characterization of the monosaccharide components of a polysaccharide by depolymerization may be by methods and techniques described in Finlayson and Du Bois, Olin Chim Acta. March 1; 84(1-2):203-6 (1978)., for example. In some embodiments, the polysaccharides produced by the modified microorganism comprise a higher number of a particular monosaccharide than the polysaccharides produced by the unmodified microorganism. In one embodiment, the particular monosaccharide is fucose. In another embodiment, the particular monosaccharide is arabinose. In yet another embodiment, the particular monosaccharide is galactose. Other embodiments describe a polysaccharide produced by a modified microorganism which comprise multiple particular monosaccharides that are present in higher number than the polysaccharide produced by the unmodified microorganism.

In some embodiments, the exocellular polysaccharides produced by the modified microorganism comprise a lower number of a particular monosaccharide than the polysaccharides produced by the unmodified microorganism. In one embodiment, the particular monosaccharide is glucose. In another embodiment, the particular monosaccharide is xylose. In yet another embodiment, the particular monosaccharide is fructose. Other embodiments describe an exocellular polysaccharide produced by a modified microorganism which comprise multiple particular monosaccharides that are present in lower number than the exocellular polysaccharide produced by the unmodified microorganism.

In some embodiments, the polysaccharides produced by the microorganisms according to the disclosure are high molecular weight polysaccharides. In one embodiment, high molecular weight polysaccharides comprise a molecular weight of at least about 300 kilodaltons (kDa), as shown in FIG. 6. In other embodiments, high molecular weight polysaccharides comprise a molecular weight of at least about 50, 100, 200, 400, 500, 600, 700, 800, 900, 1000 or more kDa. Whether a polysaccharide is considered a high molecular weight polysaccharide will depend on the species of oleaginous microorganism and the fermentation broth.

In certain embodiments where both modified and unmodified microorganisms produce high molecular weight exocellular polysaccharides, the production of high molecular weight polysaccharides by the modified microorganism is lower than the production of high molecular weight polysaccharides by the unmodified microorganism. In other embodiments, the modified microorganism produces a fermentation broth comprising a lower relative abundance of high molecular weight exocellular polysaccharides than the fermentation broth of the unmodified microorganism.

### 5. FERMENTATION BROTH VISCOSITY

The effect of exocellular polysaccharides on viscosity has been characterized previously in bacteria and algae fermentation. (de Swaff, et al., Appl Microbiol Biotechnol. October; 57(3):395-400 (2001); Becker, et al., Appl Microbiol Biotechnol. August; 50(2):145-52. (1998)). Production of exocellular polysaccharides by the microbes results in an increase in the biomass of the viscosity of the fermentation broth. High viscosity due to polysaccharide production complicates the development of high cell density fermentations, such as those required for biofuel applications. To achieve these high cell density levels, low viscosities and the resulting high mass transfer coefficients are required. Many microorganisms cannot produce these required low viscosities and the high mass transfer coefficients due to production of exocellular polysaccharide, and are thus not suited for biofuel applications.

Disclosed are modified microorganisms that produce fermentation broth with low viscosity measurements during high nutrient fermentations, allowing these microorganisms to achieve higher biomass levels for high density applications. In one aspect, the oleaginous microorganisms as disclosed produce a fermentation broth. In some embodiments, the modified microorganism produces a fermentation broth having a lower viscosity than a fermentation broth produced by the unmodified microorganism when grown in culture (Table 1 Error! Reference source not found).

Viscosity can be measured any number of ways. Viscometers are typically used, for example, such as a standard Brookfield viscometer or a capillary Cannon-Fenske routine viscometer (Schott, Mainz, Germany), or a Vismetron viscometer (manufactured by Shibaura System Co, Ltd.). Any method or device for measuring viscosity of a fermentation broth can be used.

In certain embodiments, the fermentation broth containing the modified oleaginous microorganism has a substantially similar cell density to the cell density of the fermentation broth produced by the unmodified microorganism.

The fermentation broth should comprise a minimum quantity of biomass to produce enough fatty acids. In some embodiments, the fermentation broth of each of the modified and unmodified microorganism comprises a biomass of at least about 50 grams cellular dry weight per liter. In other embodiments, the biomass of the fermentation broth of each microorganism is at least about 5, 10, 15, 20, 25, 30, 35, 40, or 45 grams per liter. In other embodiments, the biomass of the fermentation broth of each microorganism is at least about 60, 70, 80, 90, 100, 125, 150, 175, 200, 300, 400, or 500 or more grams per liter of cellular dry weight.

In one aspect, a microorganism according to this disclosure produces a fermentation broth comprising both a minimum biomass with a maximum viscosity. In certain embodiments, the modified microorganism produces a fermentation broth comprising a biomass of at least about 50 grams cellular dry weight per liter and a viscosity of less than about 1,100 centipoise (cP) (see Table 1). In other embodiments, the modified microorganism produces a fermentation broth comprising a biomass of at least about 50 grams cellular dry weight per liter and a viscosity of less than about 700 cP. In other embodiments, the modified microorganism produces a fermentation broth comprising a biomass of at least about 50 grams cellular dry weight per liter and a viscosity of less than about 100 cP. In other embodiments, the modified microorganism produces a fermentation broth comprising a biomass of at least about 50 grams cellular dry weight per liter and a viscosity of less than about 30 cP. In still other embodiments, the modified microorganism produces a fermentation broth comprising a biomass of at least about 50 grams cellular dry weight per liter and a viscosity of less than about 2.0, 2.5, 3.0, 3.5, 4.0, 4.5, 5, 6, 7, 8, 9, 10, 15, 20, or 25 cP. In yet other embodiments, the modified microorganism produces a fermentation broth comprising a biomass of at least about 50 grams cellular dry weight per liter and a viscosity of less than about 35, 40, 45, 50, 60, 70, 80, 90, 100, 200, 300, 400, 500, 600, 700, 800, 900, 1000, 1500, 2000, 2500, or 3000 cP or more.

In another aspect, the modified microorganisms as disclosed produce a fermentation broth that has a lower viscosity than the viscosity of the fermentation broth produced by the unmodified microorganisms. In some embodiments, the modified microorganism produces a fermentation broth comprising a biomass of at least about 50 grams cellular dry weight per liter and a viscosity at least about 10 times lower than the viscosity of a substantially similar fermentation broth produced by the unmodified microorganism. In other embodiments, the modified microorganism produces a fermentation broth comprising a biomass of at least about 50 grams cellular dry weight per liter and a viscosity at least about 100 times lower than the viscosity of the fermentation broth produced by the unmodified microorganism. In other embodiments, the modified microorganism produces a fermentation broth comprising a biomass of at least about 50 grams cellular dry weight per liter and a viscosity at least about 500 times lower than the viscosity of the fermentation broth produced by the unmodified microorganism. In other embodiments, the modified microorganism produces a fermentation broth comprising a biomass of at least about 50 grams cellular dry weight per liter and a viscosity at least about 2, 3, 4, 5, 6, 7, 8, 9, 15, 20, 30, 40, 50, 60, 70, 80, 90, 150, 200, 300, 400, 600, or 1000 or more times lower than the viscosity of the fermentation broth produced by the unmodified microorganism.

### 6. AGITATION POWER AND NUTRIENT AVAILABILITY

Viscosity is an important contributor to the engineering design of aerobic fermentation systems at industrial scale. A major factor in the design of industrial scale fermenters is provision for adequate mass transfer of oxygen into solution and maintenance of at least a minimum dissolved oxygen concentration. Some microorganisms in fermentation broth require oxygen supplementation to sustain adequate dissolved oxygen levels for cell survival and propagation.

In some embodiments, the modified microorganism produces a fermentation broth that can maintain a minimal dissolved oxygen (abbreviated “DO”) level without oxygen supplementation. The dissolved oxygen level can be measured by any one of several methods. One method of measuring the degree of oxygen saturation in the fermentation broth is using an oxygen probe. The probe will send a signal that indicates the amount of oxygen in the fermentation broth as a percentage relative to the calibrated maximum oxygen signal. In certain embodiments, the minimal dissolved oxygen level comprises about 20 percent. See Table 1, column 6, labeled “% DO”) In other embodiments, the minimal dissolved oxygen level comprises about 10, 15, 25, 30 percent or higher. Different species of microorganism may require various levels of dissolved oxygen for cell viability and propagation.

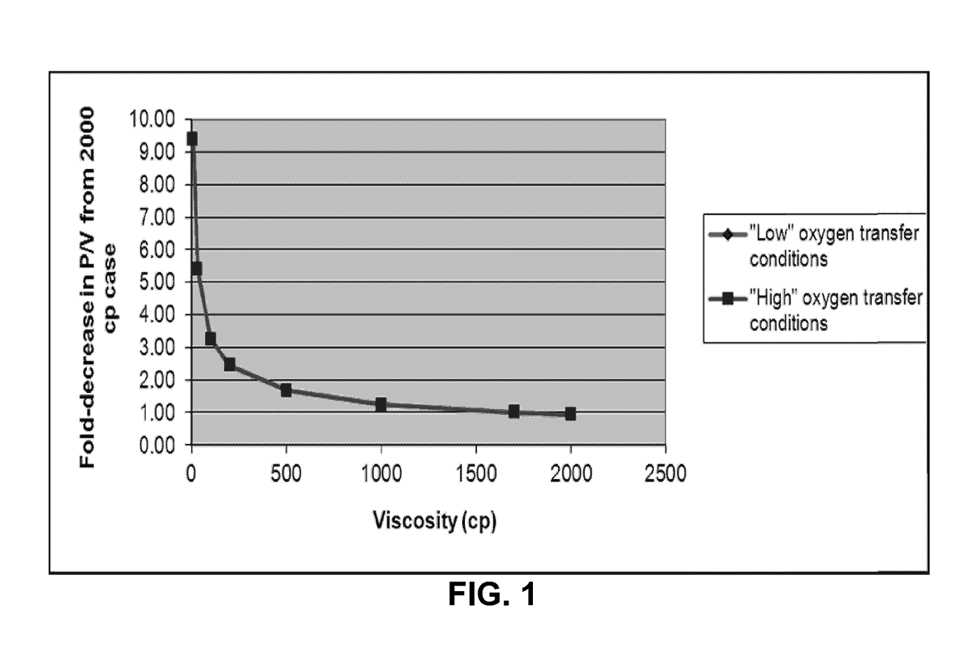

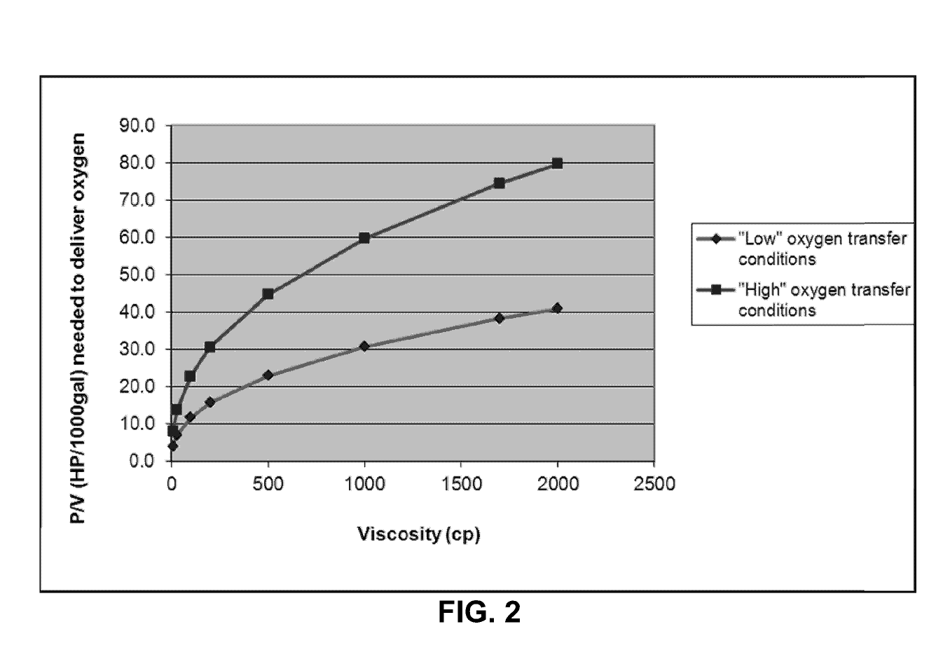

A high viscosity of culture broth increases the energy input required for mixing and may also reduce the maximum rate of oxygen transfer. For example, this has been demonstrated in xanthan-producing Xanthomonas campestris cultures, (Shu and Yang, Biotechnol Bioeng. March 5; 35(5):454-68 (1990), High viscosity fermentation broth limits mass transfer, resulting in the need for greater agitation and aeration power inputs to provide sufficient oxygen and other nutrients to the cells in fermentation broth (FIGS. 1 and 2). To maintain the same oxygen mass transfer as viscosity increases, increased delivered horsepower (such as power per volume) is required, usually by a combination of agitation and air compressor work, The requirement for increased power for agitation increases the cost of fermentation considerably.

The oxygen mass transfer coefficient is one way that the availability of oxygen in fermentation broth is calculated. The oxygen mass transfer coefficient can be calculated by one of ordinary skill in the art, and is typically calculated from plots of dissolved oxygen tension versus time (de Swaff, et al., Appl Microbiol Biotechnol. October; 57(3):395-400 (2001)). Also available are empirical correlations describing the relationship between solution viscosity (μ), oxygen mass transfer rate (kLa), superficial air velocity (Us), and delivered horsepower (P/V). For example, the most common empirical correlation is as follows:

kLa=A*(P/V)̂B*(Us)̂C*(μ)̂D

Appropriate values for the constants A, B, C, and D that represent the empirical correlation between each parameter and oxygen mass transfer coefficient (kLa) can be readily selected and/or calculated by one of ordinary skill in the art.

In certain embodiments, the modified microorganism has an oxygen mass transfer coefficient that is higher than the oxygen mass transfer coefficient of the unmodified microorganism. In other embodiments, the modified microorganism does not require oxygen supplementation when grown in culture, but the unmodified microorganism requires oxygen supplementation when grown in culture. Therefore, reducing the solution viscosity would also reduce the power per volume required to deliver oxygen and other nutrients.

Polysaccharide concentration is likewise an important contributor to the viscosity of a solution. Empirical correlations can be made between the concentrations of polysaccharide in solution with the observed solution viscosity.

In one aspect, the microorganisms as disclosed require a reduced quantity of power to agitate a unit volume of fermentation broth. The amount of power required to agitate a volume (typically measured in horsepower per 1000 gallons or kilowatt per cubic meter) of fermentation broth can be calculated by one of ordinary skill in the art. In some embodiments, the unit volume is 1000 gallons. This reduced power requirement provides for a less expensive fermentation process.

In one embodiment, the modified microorganism can be cultured in fermentation broth requiring less than 8.0 horsepower per 1000 gallons for agitation (FIG. 2). In another embodiment, the modified microorganism can be cultured in fermentation broth requiring less than 5.0 horsepower per 1000 gallons for agitation. In yet other embodiments, the modified microorganism can be cultured in fermentation broth requiring less than 4.0, 3.0, 2.0, 1.0 or less horsepower per 1000 gallons for agitation.

In another embodiment, the modified microorganism requires less agitation horsepower per unit volume than the unmodified microorganism. In certain embodiments, the modified microorganism requires at least about 9 fold less agitation horsepower per 1000 gallons than the unmodified microorganism. In other embodiments, the modified microorganism requires at least about 5, 10, 15, 20, 25, 50, 100, 1000 or higher fold less agitation horsepower per unit volume than the unmodified microorganism.

### 7. FATTY ACID YIELD

All of the microorganisms disclosed herein, both modified and unmodified, can produce a fatty acids during fermentation. Fatty acid synthesis is negatively impacted by polysaccharide production in many microorganisms. The conditions that promote lipid production (high carbon to nitrogen ratios) also promote polysaccharide production. Fatty acid fermentation yield decreases can occur in these microorganisms because part of the carbon source is utilized for polysaccharide production instead of the desired fatty acid or lipid. As viscosity of the fermentation broth increases with increasing polysaccharide quantities, there is also a reduction in mass transfer, which can reduce the efficiency of fatty acid synthesis. Fatty acid extraction processes are also negatively affected by polysaccharide production. Cell harvesting is difficult via filtration or centrifugation in the presence of polysaccharides. Cell breakage is inefficient in the presence of polysaccharides. Polysaccharides can contribute to the formation of stable emulsions. High levels of polysaccharide can also prevent oil extraction and recovery in aqueous systems.

One measure of microorganism productivity is fatty acid fermentation yield. By introducing genetic modifications into the unmodified microorganisms according to this disclosure, novel modified microorganisms were created which generally improved the fermentation yield of fatty acids on sugar (carbon substrate) by approximately 20-25 wt %. The yield of the fatty acid of the described microorganisms can be readily calculated by one of ordinary skill in the art. Typically the fatty acid methyl ester, or FAME, is assayed.

A fatty acid methyl ester (FAME) can be created by an alkali catalyzed reaction between fats or fatty acids and methanol, to produce a fuel or assay a fatty acid profile produced by a microorganism. The types and proportions of fatty acids present in the lipids of cells, or the fatty acid profile, are major phenotypic traits and can be used to identify microorganisms. For example, analysis using gas chromatograph (“GC”), can determine the lengths, bonds, rings and branches of the FAME. The primary reasons to analyze fatty acids as fatty acid methyl esters include: In their free, underivatized form, fatty acids may be difficult to analyze because these highly polar compounds tend to form hydrogen bonds, leading to adsorption issues, Reducing their polarity may make them more amenable for analysis. To distinguish between the very slight differences exhibited by unsaturated fatty acids, the polar carboxyl functional groups can be first be neutralized. This then allows column chemistry to perform separations by boiling point elution, and also by degree of unsaturation, position of unsaturation, and even the cis versus trans configuration of unsaturation.

The esterification of fatty acids to fatty acid methyl esters may be performed using an alkylation derivatization reagent. Methyl esters offer excellent stability, and provide quick and quantitative samples for GC analysis. The esterification reaction involves the condensation of the carboxyl group of an acid and the hydroxyl group of an alcohol. Transesterification can include use of any suitable alcohol, such as methanol, ethanol, propanol, butanol, and/or the like. Esterification can be done in the presence of a catalyst (such as boron trichloride). The catalyst protonates an oxygen atom of the carboxyl group, making the acid much more reactive. An alcohol then combines with the protonated acid to produce an ester with the loss of water. The catalyst is removed with the water. The alcohol that is used determines the alkyl chain length of the resulting esters (the use of methanol will result in the formation of methyl esters whereas the use of ethanol will result in ethyl esters).

In most embodiments, the disclosed modified microorganisms comprise a fatty acid fermentation yield that is greater than the fatty acid fermentation yield of the unmodified microorganism. In certain embodiments, the modified microorganism exhibits a fatty acid fermentation yield of at least about 14 percent. In other embodiments, the modified microorganism has a fatty acid fermentation yield of at least about 5, 10, 15, 20, 21, 22, 23, 24, 25, 26, 27, 28, 29, 30, 31, 32, 33, 34, 35 percent or higher (Table 1).

In some embodiments, the modified microorganism produces a fatty acid fermentation yield at least about 10 percent greater than the fermentation yield of the fatty acid produced by the unmodified microorganism. In other embodiments, the modified microorganism produces a fatty acid yield at least about 20 percent greater than the yield of the fatty acid produced by the unmodified microorganism. In yet other embodiments, the modified microorganism produces a fatty acid yield about 10 percent to about 30 percent greater than the yield of the fatty acid yield produced by the unmodified microorganism. In further embodiments, the modified microorganism produces a fatty acid yield at least about 30, 40, 50, 100, 200, 500, 1000 or higher percent greater than the yield of the fatty acid produced by the unmodified microorganism.

### 8. BIOFUEL PRODUCTION

This disclosure also includes production of Microbial lipids and production of biofuel and/or biofuel precursors using the fatty acids contained in those lipids. This disclosure provides for microorganisms that produce lipids suitable for biodiesel production and/or nutritional applications at a very low cost.

According to some embodiments, the disclosure can include a method of producing biological oils. The method can include producing or growing a microorganism as disclosed herein. The microorganism can include and/or have within a lipid containing fatty acids and/or a quantity of lipids containing fatty acids. In the alternative, the organism can excrete and/or discharge the biological oil.

The method can further include any suitable additional actions, such as extracting and/or removing the lipid containing fatty acids by cell lysing, applying pressure, solvent extraction, distillation, centrifugation, other mechanical processing, other thermal processing, other chemical processing, and/or the like. In the alternative, the producing microorganism can excrete and/or discharge the lipid containing fatty acids from the microorganism without additional processing.

The fatty acids can have any suitable profile and/or characteristics, such as generally suitable for biofuel production. According to some embodiments, the fatty acids can include a suitable amount and/or percent fatty acids with four or more double bonds on a mass basis. In the alternative, the fatty acids can include a suitable amount and/or percent fatty acids with three or more double bonds, with two or more double bonds, with one or more double bonds, and/or the like.

In another aspect, disclosed are methods of producing a biofuel precursor. In certain embodiments, the methods comprise culturing the microorganisms as described and collecting the fermentation broth produced by the microorganism. The biofuel precursor can be produced using any of the microorganisms described herein. In some embodiments, the biofuel precursor is a biological oil. The biofuel precursor can be extracted as described herein or by any other suitable technique. If necessary, further chemical processing of extracted lipids and/or biological oils into biofuel precursors can be performed. In some embodiments, the method further comprises extracting fatty acids from the microorganism and reacting the fatty acids to produce a biofuel.

Also disclosed are methods for producing a biofuel. In certain embodiments, the method comprises supplying a carbon source and converting the carbon source to fatty acids within the microorganisms as described. Certain described microorganisms should be cultured to a specific cell density prior to extraction of lipids, oils, biofuels, or biofuel precursors. In certain embodiments, the disclosed method comprises culturing the microorganism to a cell density of at least about 50 grams cellular dry weight per liter in a fermentation broth having a viscosity of less than about 1100 cP. In one embodiment, the biofuels or biofuel precursors of the method is produced with any of the modified microorganism as disclosed herein, In one embodiment, the microorganism is exocellular polysaccharide-producing yeast. In other embodiments, the disclosed method comprises culturing the microorganism to a cell density of at least about 10, 20, 30, 40, 60, 70, 80, 90, 100, 200, 300, 400, 500, 1000 or more grams per liter in a fermentation broth having a viscosity of less than about 1500, 1000, 750, 500, 100, 50, 30, 10, 5 or lower cP.

A biofuel produced by the described methods is also described. The biofuel may be derived from any of the biofuel precursors or biological oils or lipids as produced by the disclosed methods or microorganisms. The biofuel precursor or biological oil can be further processed into the biofuel with any suitable method, such as esterification, transesterification, hydrogenation, cracking, and/or the like. In the alternative, the biological oil can be suitable for direct use as a biofuel. Esterification refers to making and/or forming an ester, such as by reacting an acid with an alcohol to form an ester. Transesterification refers to changing one ester into one or more different esters, such as by reaction of an alcohol with a triglyceride to form fatty acid esters and glycerol, for example. Hydrogenation and/or hydrotreating refer to reactions to add hydrogen to molecules, such as to saturate and/or reduce materials.

In another aspect, disclosed are methods of powering a vehicle by combusting a biofuel in an internal combustion engine. The biofuel can be produced by any of the described methods or by any of the disclosed microorganisms.

In another aspect, disclosed is a biofuel suitable for use in compression engines. The biofuel can be produced by any of the described methods or by any of the disclosed microorganisms.

Increasing interest is directed to the use of hydrocarbon components of biological origin in fuels, such as biodiesel, renewable diesel, and jet fuel, since renewable biological starting materials that may replace starting materials derived from fossil fuels are available, and the use thereof is desirable. There is an urgent need for methods for producing hydrocarbon components from biological materials, The present disclosure fulfills this need by providing methods and microorganisms suited for production of biodiesel, renewable diesel, and jet fuel using the lipids generated by the methods described herein as a biological material to produce biodiesel, renewable diesel, and jet fuel.

After extraction, lipid and/or hydrocarbon components recovered from the microbial biomass described herein can be subjected to chemical treatment to manufacture a fuel for use in diesel vehicles and jet engines. One example is that biodiesel can be produced by transesterification of triglycerides contained in oil-rich biomass. Lipid compositions can be subjected to transesterification to produce long-chain fatty acid esters useful as biodiesel. Thus, in another aspect of the present disclosure a method for producing biodiesel is provided. In a certain embodiment, the method for producing biodiesel comprises the steps of (a) cultivating a lipid-containing microorganism using methods disclosed herein (b) lysing a lipid-containing microorganism to produce a lysate, (c) isolating lipid from the lysed microorganism, and (d) transesterifying the lipid composition, whereby biodiesel is produced. Transesterification can include use of any suitable alcohol, such as methanol, ethanol, propanol, butanol, and/or the like.

Methods for growth of a microorganism, lysing a microorganism to produce a lysate, treating the lysate in a medium comprising an organic solvent to form a heterogeneous mixture and separating the treated lysate into a lipid composition have been described above and can also be used in the method of producing biodiesel.

The common international standard for biodiesel is EN 14214 (November 2008). Germany uses DIN EN 14214 and the UK requires compliance with BS EN 14214. ASTM D6751 (November 2008) is the most common biodiesel standard referenced in the United States and Canada. Basic industrial tests to determine whether the products conform to these standards typically include gas chromatography, HPLC, and others. Biodiesel meeting the quality standards is very non-toxic, with a toxicity rating of greater than 50 mL/kg, The resulting biofuel can meet and/or exceed international standards EN 14214:2008 (Automotive fuels, Fatty acid methyl esters (FAME) for diesel engines) and/or ASTM D6751-09 (Standard Specification for Biodiesel Fuel Blend Stock (B100) for Middle Distillate Fuels). The entire contents of EN 14214:2008 and ASTM D6751-09 are hereby both incorporated by reference in their entirety as a part of this specification.

### 9. RENEWABLE MATERIAL PRODUCTION

The production of renewable materials, including biological oils, from sources such as plants (including oilseeds), microorganisms, and animals needed for various purposes. For example, it is desirable to increase the dietary intake of many beneficial nutrients found in biological oils. Particularly beneficial nutrients include fatty acids such as omega-3 and omega-6 fatty acids and esters thereof. Because humans and many other animals cannot directly synthesize omega-3 and omega-6 essential fatty acids, they must be obtained in the diet Traditional dietary sources of essential fatty acids include vegetable oils, marine animal oils, fish oils and oilseeds. In addition, oils produced by certain microorganisms have been found to be rich in essential fatty acids. In order to reduce the costs associated with the production of beneficial fatty acids for dietary, pharmaceutical, and cosmetic uses, there exists a need for a low-cost and efficient method of producing biological oils containing these fatty acids.

In certain embodiments, the oleaginous microorganism produces a renewable material. The renewable materials as disclosed herein can be used for the manufacture of a food, supplement, cosmetic, or pharmaceutical composition for a non-human animal or human. Renewable materials can be manufactured into the following non-limiting examples: food products, pharmaceutical compositions, cosmetics, and industrial compositions. In certain embodiments, the renewable material is a biofuel or biofuel precursor.

A food product is any food for animal or human consumption, and includes both solid and liquid compositions. A food product can be an additive to animal or human foods, and includes medical foods. Foods include, but are not limited to, common foods; liquid products, including milks, beverages, therapeutic drinks, and nutritional drinks; functional foods; supplements; nutraceuticals; infant formulas, including formulas for pre-mature infants; foods for pregnant or nursing women; foods for adults; geriatric foods; and animal foods. In some embodiments, the microorganism, renewable material, or other biological product disclosed herein can be used directly as or included as an additive within one or more of: an oil, shortening, spread, other fatty ingredient, beverage, sauce, dairy-based or soy-based food (such as milk, yogurt, cheese and ice-cream), a baked good, a nutritional product, e.g., as a nutritional supplement (in capsule or tablet form), a vitamin supplement, a diet supplement, a powdered drink, a finished or semi-finished powdered food product, and combinations thereof.

In certain embodiments, the renewable material is a biological oil. In certain embodiments, the renewable material is a saturated fatty acid. Non-limiting of saturated fatty acids include oleic acid, linoleic acid, or palmitic acid.

The modified oleaginous microorganisms described herein can be highly productive in generating renewable materials as compared to unmodified counterpart microorganisms. Microorganism renewable material productivity is disclosed in pending U.S. patent application Ser. No. 13/046,065 (Pub. No. 20120034190, filed Mar. 11, 2011), which is herein incorporated by reference in its entirety. In other embodiments, the application discloses methods of producing renewable materials. Methods of producing renewable materials is disclosed in pending U.S. patent application Ser. No. 13/046,065 (Pub. No. 20120034190, filed Mar. 11, 2011), which is herein incorporated by reference in its entirety. Each reference cited in this disclosure is hereby incorporated by reference as if set forth in its entirety.

### EXAMPLES

The following examples are offered to illustrate, but not to limit, the claimed invention.

### Example 1

### Strain Mutagenesis

The strains selected for mutagenesis work were MK29404, a strain of the yeast species Sporidiobolus pararoseus, and MK29794, a strain of the yeast species Rhodotorula ingeniosa. MK29404 and MK29794 produce high viscosity broth after fermentation for about 70-100 hours, as shown in Table 1. MK28428 has a lower viscosity after comparable fermentation times (Table 1).

Genetic modifications were introduced into these strains by standard UV light, X-Ray irradiation and chemical mutagenesis. To determine the appropriate level of exposure to the different mutagens, kill curves were conducted on each strain and each mutagen. UV light, X-ray irradiation and a chemical mutagen (nitrosoguanidine) were used for each strain.

Briefly, cells were plated onto agar media plates and exposed to a range a UV irradiation dose of 350-475 μjoules. X-ray mutagenesis was conduct by plating cells onto agar media plates and exposing them to X-ray irradiation for 30 min or 1 hour. Chemical mutagenesis was conducted by mixing cells of MK29404 with varying levels of nitrosoguanidine for 1 hour. Levels of 20 and 40 μg/ml were used for subsequent generation of mutants.

Mutagenized cells were grown on agar plates with standard Biofuels Growth Media (BFGM) concentration of 1/16 of the full strength media. It was decided to utilize a BFGM concentration of 1/16 of the full strength media. This concentration allowed significant fat accumulation but prevented the colonies from overgrowing and merging together.

### Example 2

### Selection of Dry Strain Morphology

The first screen of mutant strains of MK29404 and MK29794 was visual inspection. Mutant colonies that had low levels of polysaccharide were identified based on visual observation of colonies on agar plates. Wild type colonies are ‘wet’ and ‘goopy’ in appearance and very soft. If the agar plate is upside down the colony will ‘drip’ onto the other side of the plate. This morphology is characteristic of cells that produce large amounts of extra-cellular polysaccharide. Low polysaccharide mutants were identified by a colony morphology that was “dry.” These colonies were not visibly wet or goopy, but stiff and powdery. Colonies that were “dry” were selected for further study.

### Example 3

### Fermentation of Selected Strains

Colonies with the “dry” morphology were saved for more detailed analysis and commonly referred to as “dry” mutants. Multiple strains of the mutant and wild type (WT) MK29404, MK28428, and MK29794 strains were fermented, with the WT strains representing exemplary unmodified microorganisms. Unless specified otherwise within this specification, the fermentation protocol was generally followed along or conducted according to procedures from U.S. Pat. No. 6,607,900, hereby incorporated by reference.

Each strain was cultivated in a 100 liter New Brunswick Scientific (Edison, N.J., U.S.A.) BioFlo 6000 fermentor with a carbon (glucose) and nitrogen) (ammonium hydroxide) fed-batch process. The fermentation was inoculated with 6 liters of culture. For inoculum propagation a 14 liter VirTis (SP Scientific Gardiner, N.Y., U.S.A.) fermentor was utilized. The inoculum medium included 10 liters of medium prepared in four separate groups. Group A included 98 grams MSG*1H₂O, 202 grams Na₂SO₄, 5 grams KCl, 22.5 grams MgSO₄*7H₂O, 23.1 grams (NH₄)₂SO₄, 14.7 grams KH₂PO₄, 0.9 grams CaCl₂*2H₂O, 17.7 milligrams MnCl₂*4H₂O, 18.1 milligrams ZnSO₄*7H₂O, 0.2 milligrams CoCl₂*6H₂O, 0.2 milligrams Na₂MoO₄*2H₂O, 11.8 milligrams CuSO₄*5H₂O, 11.8 milligrams NiSO₄*6H₂O, and 2 milliliters Dow (Midland, Mich., U.S.A.) 1520US (antifoam). Group A was autoclaved at 121 degrees Celsius in the inoculum fermentor at a volume of approximately 9.5 liters. Group B included 20 milliliters of a one liter stock solution containing 2.94 grams FeSO₄*7H₂O and 1 grams citric acid. The group B stock solution was autoclaved at 121 degrees Celsius. Group C included 37.6 milligrams thiamine-HCl, 1.9 milligrams vitamin B12, and 1.9 milligrams pantothenic acid hemi-calcium salt dissolved in 10 milliliters and filter sterilized. Group D included 1,000 milliliters of distilled water containing 400 grams glucose powder. After the fermentor was cooled to 29.5 degrees Celsius, groups B, C, and D were added to the fermentor. Using sodium hydroxide and sulfuric acid, the fermentor was pH adjusted to 5.5 and the dissolved oxygen was spanned to 100 percent prior to inoculation.

The inoculum fermentor was inoculated with 18 milliliters of a standard shake flask culture and cultivated at 29.5 degrees Celsius, pH 5.5, 350 revolutions per minute agitation, and 8 liters per minute of air for a period of 27 hours, at which point 6 liters of inoculum broth were transferred to the 100 liter fermentor. The 100 liter fermentor included 80 liters of fermentation media. The fermentation media was prepared in a similar fashion to the inoculum fermentor.

The fermentation media included 7 batched media groups. Group A included 1,089.6 grams Na₂SO₄, 57.6 grams K₂SO₄, 44.8 grams KCl, 181.6 grams MgSO₄*7H₂O, and 90.4 grams KH₂PO₄. Group A was steam sterilized at 122 degrees Celsius for 60 minutes in the 100 liter fermentor at a volume of approximately 35 liters. Group B included 90.4 grams (NH₄)₂SO₄ and 10.4 grams MSG*1H₂O in a volume of approximately 500 milliliters. Group C included 15.2 grams CaCl₂*2H₂O in a volume of approximately 200 milliliters. Group D included 1,200 grams of powdered glucose in approximately 2 liters of distilled water. Group E included 248 milligrams MnCl₂*4H₂O, 248 milligrams ZnSO₄*7H₂O, 3.2 milligrams CoCl₂*6H₂O, 3.2 milligrams Na₂MoO₄*2H₂O, 165.6 milligrams CuSO₄*5H₂O, and 165.6 milligrams NiSO₄*6H₂O in a volume of approximately 1 liter. Group F included 824 milligrams FeSO₄*7H₂O and 280.3 milligrams citric acid in a volume of approximately 280 milliliters. Group G included 780 milligrams thiamine-HCl, 12.8 milligrams vitamin B12, and 266.4 milligrams pantothenic acid hemi-calcium salt filter sterilized in a volume of approximately of 67.4 milliliters distilled water. Groups B, C, D, E, F, and G were combined and added to the fermentor after the fermentor reached an operating temperature of 29.5 degrees Celsius. The fermentor volume prior to inoculation was approximately 38 liters.

The fermentor was inoculated with 6 liters of broth from the fermentation described above. The fermentation was pH controlled utilizing a 5.4 liter solution of 4N ammonium hydroxide at a pH of 5.5. The dissolved oxygen was controlled between 5 percent and 20 percent throughout the fermentation using agitation from 180 revolutions per minute to 480 revolutions per minute and airflow from 60 liters per minute to 100 liters per minute. Throughout the fermentation, 38.4 liters of an 850 grams cellular dry weight per liter solution of 95 percent dextrose was fed to maintain a concentration less than 50 grams cellular dry weight per liter.

### Example 4

### Viscosity Measurements

The viscosity of each strain was assayed after a set fermentation time period, generally 50-100 hours. Culture viscosity was determined with a standard Brookfield viscometer (Middleboro, Mass.). The media components did not significantly influence the viscosity at the concentrations used.

The Dry strains showed dramatically improved viscosity measurements and improved carbon utilization. The data summarizing viscosity measurements of the unmodified wild type (WT) and Dry strains of MK 29404, MK28428, and MK 29794 are in Table 1. Mass calculations were performed in non-recycled volumes (“RV”). Average viscosity for MK29404 wild type was 1701 cP, while the average viscosity for the MK29404 Dry1 mutant was 8.5 cP, which is a 200 fold reduction in viscosity. Other MK 29404 Dry mutants had similar reductions in viscosity. The MK 29794 wild type stain had a viscosity of about 700 cP, while the Dry mutants were mostly <50 cP. Thus, the Dry mutants of the MK 29404 and 29794 showed a substantial decrease in viscosity as compared to their WT (Wild Type or unmodified) counterparts. MK28428 strains showed a low viscosity, but since the MK 29404 and MK 29794 Dry mutants displayed better measures of productivity such as fatty acid yield on sugar, the MK 28428 strains were not selected for follow-up experiments.

TABLE 1
Viscosity, oxygen supplementation, and sugar yield of generated yeast strains.

| | | | | | | FA Yield | Non-RV Cell | Non-RV Fat (g/L) | |
|---|---|---|---|---|---|---|---|---|---|
| Exp. | | Viscosity | O2 | Over | | on sugar | Density (g/L) | (TOTAL LIPID | % Fat |
| No. | Strain | (cP) | Supp. | 30 cp | % DO | (wt %) | (TOTAL MASS) | MASS) | (FAME) |
| | UNMODIFIED | | | | | | | | |
| 1 | MK29404 | 1138 | yes | yes | 20 | 17.8 | 113.4 | 61.1 | 53.9 |
| 2 | MK29404 | 1160 | yes | yes | 20 | 12.9 | 140.7 | 66.1 | 47.0 |
| 3 | MK29404 | 1180 | yes | yes | 20 | 14.1 | 144.9 | 69.8 | 48.1 |
| 4 | MK29404 | 1222 | yes | yes | 20 | 19.8 | 104.1 | 55.5 | 53.4 |
| 5 | MK29404 | 1309 | yes | yes | 20 | 15.2 | 111.8 | 55.9 | 50.0 |
| 6 | MK29404 | 1350 | yes | yes | 20 | 17.0 | 104.1 | 55.9 | 53.8 |
| 7 | MK29404 | 1470 | yes | yes | 20 | 14.8 | 109.1 | 54.3 | 49.8 |
| 8 | MK29404 | 1512 | yes | yes | 20 | 15.1 | 109.5 | 51.8 | 47.3 |
| 9 | MK29404 | 1831 | yes | yes | 20 | 17.3 | 115.4 | 62.8 | 54.4 |
| 10 | MK29404 | 1932 | yes | yes | 20 | 16.8 | 118.1 | 60.4 | 51.2 |
| 11 | MK29404 | 1974 | yes | yes | 20 | 18.2 | 120.6 | 62.1 | 51.5 |
| 12 | MK29404(Pale1) | 1512 | yes | yes | 20 | 18.3 | 109.2 | 48.4 | 44.3 |
| 13 | MK29404(Pale1) | 1751 | yes | yes | 20 | 20.4 | 110.6 | 54.4 | 49.2 |
| 14 | MK29404 | 1722 | yes | yes | 20 | 16.9 | 122.9 | 61.7 | 50.2 |
| 15 | MK29404 | 1974 | yes | yes | 20 | 18.8 | 127.3 | 66.2 | 52.0 |
| 16 | MK29404 | 2142 | yes | yes | 20 | 19.2 | 116.5 | 63.2 | 54.3 |
| 17 | MK29404 | 2226 | yes | yes | 20 | 11.1 | 78.7 | 50.0 | 63.5 |
| 18 | MK29404 | 2310 | yes | yes | 20 | 16.6 | 113.6 | 56.7 | 49.9 |
| 19 | MK29404 | 2478 | yes | yes | 20 | 11.0 | 92.5 | 33.3 | 36.0 |
| 20 | MK28428 | 39.8 | no | yes | 20 | 14.2 | 23.6 | 11.9 | 50.5 |
| 21 | MK28428 | 40.5 | no | yes | 20 | 14.2 | 68.8 | 34.6 | N/A |
| 22 | MK28428 | 40.5 | no | yes | 20 | 15.8 | 74.0 | 36.6 | 49.5 |
| 23 | MK28428 | 40.5 | no | yes | 20 | 13.6 | 65.7 | 31.6 | 48.1 |
| 24 | MK28428 | 5.22 | no | no | 20 | 17.0 | 129.0 | 63.2 | 49.0 |
| 25 | MK29794 | 718.6 | yes | yes | 20 | 15.8 | 121.9 | 51.2 | 42.0 |
| | DRY MUTANTS | | | | | | | | |
| 26 | MK29404(Dry1-13J) | 1.8 | no | no | 20 | 17.6 | 7.4 | 1.4 | 18.9 |
| 27 | MK29404(Dry1) | 2.21 | no | no | 20 | 20.9 | 133.1 | 81.4 | 61.2 |
| 28 | MK29404(Dry1) | 2.69 | no | no | 20 | 20.0 | 138.4 | 81.5 | 58.9 |
| 29 | MK29404(Dry1) | 2.72 | no | no | 20 | 20.3 | 140.0 | 81.6 | 58.3 |
| 30 | MK29404(Dry1-147D) | 4.59 | no | no | 20 | 11.3 | 83.4 | 29.3 | 35.2 |
| 31 | MK29404(Dry1-72D) | 5.52 | no | no | 20 | 15.8 | 103.1 | 51.6 | 50.0 |
| 32 | MK29404 (Dry-1) | 5.91 | no | no | 20 | 13.4 | 100.0 | 43.5 | 43.6 |
| 33 | MK29404(Dry1-182J) | 6.03 | no | no | 20 | 15.7 | 112.6 | 59.0 | 52.4 |
| 34 | MK29404(Dry1) | 6.39 | no | no | 20 | 18.7 | 119.7 | 69.7 | 58.2 |
| 35 | MK29404 (Dry-1) | 7.14 | no | no | 20 | 21.0 | 127.3 | 72.7 | 57.1 |
| 36 | MK29404(Dry1-173N) | 7.14 | no | no | 20 | 15.8 | 116.4 | 65.8 | 56.6 |
| 37 | MK29404(Dry55) | 7.71 | no | no | 20 | 17.3 | 117.8 | 65.9 | 55.9 |
| 38 | MK29404(Dry41) | 8.97 | no | no | 20 | 16.2 | 103.8 | 50.3 | 48.5 |
| 39 | MK29404 (Dry-1) | 9.81 | no | no | 20 | 19.4 | 147.7 | 85.6 | 57.9 |
| 40 | MK29404 Dry-1 | 11 | no | no | 20 | 19.4 | 133.8 | 84.3 | 63.0 |
| 41 | MK29404 (Dry-1) | 11.5 | no | no | 20 | 15.7 | 131.2 | 79.8 | 60.8 |
| 42 | MK29404 (Dry-1) | 23.5 | no | no | 20 | 20.1 | 144.2 | 82.2 | 57.0 |
| 43 | MK29404 (Dry1) | <42 | yes | unknown | 20 | 18.1 | 131.3 | 78.7 | 59.9 |
| 44 | MK29404 (Dry1) | <42 | yes | unknown | 20 | 18.9 | 144.7 | 84.8 | 58.6 |
| 45 | MK29404 (348 dry) | 2369 | yes | yes | 20 | 14.8 | 99.3 | 45.1 | 45.4 |
| 46 | MK28428 (3ZA-LF) | 5.52 | yes | no | 20 | 18.9 | 115.3 | 62.9 | 54.6 |
| | | | (partial) | | | | | | |
| 47 | MK28428 (3Z-LF) | 6.51 | yes | no | 20 | 17.7 | 112.3 | 59.0 | 52.6 |
| 48 | MK28428 (163Z-LF) | 5.94 | no | no | 20 | 16.3 | 100.9 | 45.2 | 44.9 |
| 49 | MK28428 (477H) | 5.91 | no | no | 20 | 16.5 | 146.4 | 84.7 | 57.9 |
| 50 | MK28428 (156I) | 9.43 | no | no | 20 | 18.3 | 125.8 | 63.4 | 50.4 |
| 51 | MK28428 (8-500-3A) | 10.4 | no | no | 20 | 14.2 | 101.4 | 52.4 | 51.6 |
| 52 | MK28428 (155A) | 10 | no | no | 20 | 19.9 | 127.3 | 69.5 | 54.6 |
| 53 | MK28428 (163Z-LF) | 5.88 | no | no | 20 | 16.9 | 119.1 | 57.0 | 47.9 |
| 54 | MK29794 (K200Dry1) | 5.4 | yes | no | 20 | 17.4 | 98.2 | 31.4 | 32.0 |
| 55 | MK29794 (33Dry1) | 33.4 | yes | yes | 20 | 14.9 | 107.3 | 48.9 | 45.5 |
| | | | (partial) | | | | | | |
| 56 | 29794 (K200Dry) | 51 | yes | yes | 20 | 20.0 | 134.0 | 75.2 | 56.1 |
| 57 | MK29794 (33Dry) | 323.7 | yes | yes | 20 | 18.9 | 127.4 | 67.8 | 53.2 |
| 58 | MK29794 (KDry) | 7.5 | no | no | 20 | 19.8 | 135.2 | 80.5 | 59.5 |
| 59 | MK29794 (K200Dry1) | 32.2 | yes | yes | 20 | 19.5 | 122.1 | 67.4 | 55.2 |
| 60 | MK29794 (KDry16-1) | 13.4 | yes | no | 20 | 13.8 | 85.0 | 44.1 | 51.9 |
| 61 | MK29794 (117D) | 2859 | yes | yes | 20 | 16.3 | 115.3 | 56.8 | 49.3 |

### Example 5

### Dry Mass FAME Measurements

FAME analysis is described herein, but not limited to this disclosure. Briefly, lipid produced is measured by sampling the fermentation broth at the end of the fermentation, and isolating by centrifugation the lipid-containing yeast cells. The water is removed and the lipid inside the cells are converted to esters using an analytical acid-catalysed esterification protocol. Once the internal lipids are esterified to FAME, they are analyzed by gas chromatograph with an internal reference standard in order to quantify the amount of lipids recovered. The FAME analysis at this step was performed on all strains tested, as shown in Table 1. In general, MK29404 and MK29794 Dry mutants on average showed a higher FAME percentage than their unmodified wild type (WT) counterparts.

As a measure of FAME production normalized across the different strains, the sugar yield of the unmodified WT and Dry mutant strains were assayed. The sugar yield was measured by calculating the total amount of sugar consumed by the organism relative to the amount of lipid produced by the organism. Thus, sugar yield is calculated by the sum of the mass of the FAME produced divided by the sum of the mass of sugar consumed. Sugar consumed by the organism is measured by HPLC analysis of all feed-sugar solutions and totaling the volume of sugar solutions fed during the fermentation. HPLC samples are also taken just prior to the start of fermentation and just after the completion of fermentation in order to verify the amount of sugar in the starting inoculum and the amount of unconsumed sugar remaining after fermentation.

Fatty acid sugar yield results for all strains are presented in Table 1, column 7. Generally, Dry mutants had an improved sugar yield than the WT strains, improving by approximately 20-25%. For example, the wild type 29404 had an average sugar conversion yield of 16.1% compared to Strain 29404-Dry1 which had an average of 19.2%, which is about a 20% improvement. The wild type strain of 29794 had a sugar yield of 15.1%, while the 33Dry1 and KDry7 had yield percentages of 18.0 and 18.9, which was an improvement of up to 25%.

### Example 6

### Oxygen Supplementation Measurement

The oxygen supplementation requirement of all of the strains were tested. Oxygen levels in the fermentation broth were measured using an Oxygen Sensor DT222A (Fourier, Mokena, Ill.) at various time points during the fermentation, If the oxygen levels fell below 20% threshold, the strain was determined to require oxygen supplementation to support cell growth.

Prior to fermentation, the oxygen probe was calibrated. At the very start of the fermentation, there is an oxygen probe in the tank and air is blown into the vessel at max aeration and agitation, simulating maximum oxygen saturation (“100% oxygen”). For the rest of the fermentation, the probe will continue to send a 4-20 mA signal that indicates the amount of oxygen in the tank relative to the 100% signal.

The fermentation controller will adjust both the aeration rate (room air) and the agitation speed in order to maintain 20% dissolved oxygen (“DO”) (20% of the 100% signal), When oxygen supplementation is required, that indicates that in order to achieve 20% dissolved oxygen, pure oxygen had to be used rather than room air, which contains 21% oxygen.

If a strain requires oxygen supplementation, this indicates that the mass transfer is poor due to high viscosity in the strains. As evidence of improvements in mass transfer characteristics in the low viscosity strains, Table shows that high viscosity strains consistently required oxygen supplementation to maintain the desired dissolved oxygen level of 20%. The low viscosity mutants of MK29404 consistently did not require oxygen supplementation. While many of the MK29794 low viscosity mutants still required oxygen supplementation, there were mutants found that did not, such as the MK29794 KDry mutant.)

### Example 7

### Agitation Power Requirements

Strains with high viscosity require higher power inputs to the agitator motor and aeration pumps. The power per volume (P/V) was calculated as follows: For the low oxygen transfer conditions, the P/V was measured to achieve kla of 0.041 sec-1 (and an associated average OUR of 45 mmol/l/h). For the high oxygen transfer conditions, P/V was measured to achieve kla of 0.100 sec-1 (and an associated average OUR of 100 mmol/l/h). The power per volume requirement was measured and correlated with the viscosities of the broth, as shown in Table 2. These values were used to generate the graphs as shown in FIGS. 1 and 2, which illustrates the dramatic effect of viscosity on fermentation broth agitation requirements.

TABLE 2
Increased power per volume (P/V) requirements as viscosity increases.

| | |
|---|---|
| Viscosity | P/V |
| (cp) | (HP/1000 gal) |

Low Oxygen Transfer Conditions

| | | |
|---|---|---|
| | | Fold-decrease in P/V from |
| | | 2000 cp case |
| 8 | 4.1 | 9.36 |
| 30 | 7.1 | 5.38 |
| 100 | 11.8 | 3.26 |
| 200 | 15.7 | 2.44 |
| 500 | 23.0 | 1.67 |
| 1000 | 30.7 | 1.25 |
| 1700 | 38.3 | 1.00 |
| 2000 | 41.0 | 0.93 |

High Oxygen Transfer Conditions

| | | |
|---|---|---|
| | | Fold-decrease in P/V from |
| | | 1700 cp case |
| 8 | 7.9 | 9.38 |
| 30 | 13.8 | 5.39 |
| 100 | 22.9 | 3.26 |
| 200 | 30.5 | 2.44 |
| 500 | 44.7 | 1.67 |
| 1000 | 59.7 | 1.25 |
| 1700 | 74.5 | 1.00 |
| 2000 | 79.7 | 0.93 |

### Example 8

### Isolation and Quantification of Exocellular Polysaccharide

In order to investigate the source of the reduced viscosity, the exocellular polysaccharide produced by MK29404 Dry-1 was isolated and analyzed. The polysaccharide produced by the MK29404 wild type (WT) strain was also analyzed to determine the differences, if any. The polysaccharide was isolated after these strains were grown under conventional high volume (10 L) fermentation conditions as well as grown in low volume (250 ml) shaker flasks.

For the high volume fermentation experiment, strains were grown in a 10 L fermenter as described herein. The MK29404 WT strain was grown using standard media in a NBS11 vessel, according to the following conditions: T154 1.0, pH 7.0, temperature 27° C., NH₄OH feed 11.8 and carbon feed sucrose. The MK29404 Dry-1 mutant strain was grown in a NBS33 vessel using Raceland Defined Media, comprising 1.25×N&P, deleted tastone (adj. for N, P, biotin, metals, vitamins), deleted thiamine and vitamin B12 and all metals (except Fe, citric acid, Zn) [double biotin/panthenate, 2.5×], 1.2465 g/L of citric acid. Under the high volume conditions, at harvest, the viscosity of the MK29404 WT was 1700 cP. The viscosity of the MK29404 Dry-1 was 8.0 cP (Table 1).

To quantify the polysaccharide, the crude polysaccharide was subjected to isolation and purification from the culture supernatant of a batch cultivation of microorganism. For a more detailed protocol, see De Swaff et al; Miyazaki & Yamada, J. Gen. Microbio. 95, 31-38(1976). To isolate the polysaccharide from the high volume fermentation, 15 g of whole broth was weighed out. The whole broth was diluted with 25 g water and 10 g of chloroform, vortexed, and centrifuged at 4500 g for 15 min. One 10 mL aliquot of aqueous supernatant is pipetted out. 40 mL of ethanol is added to this aliquot to precipitate polysaccharide. The precipitated polysaccharide is centrifuged at 4500 g for 5 min. The supernatant is decanted, and the polysaccharide remains as pellet. The polysaccharide is resuspended in water, and the ethanol precipitation is repeated, followed by the centrifugation and decanting steps. Polysaccharide is dried down using with nitrogen stream. The net mass of crude polysaccharide is then measured and can be extrapolated as shown in Table 3. For example, the approximate total polysaccharide concentration in the initial aliquot can then be calculated by multiplying the purity factor by the net polysaccharide mass obtained from isolation procedure. The other calculations are readily understood by one of ordinary skill in the art.

In the low volume shaker flask experiment, both the MK29404 WT and the MK29494 Dry-1 mutant strains were grown with three-quarters BFGM with enriched nitrogen and phosphorous. The carbon feed for both strains was sucrose. Under the low volume growth conditions, at harvest, the viscosity of the MK29404 WT was 4.11 cP. The viscosity of the rv1K29404 Dry-1 was 1.68 cP (Table 3).

TABLE 3
Polysaccharide quantification experiments under different
fermentation conditions and volume.

| | Final Polysaccharide | |
|---|---|---|
| Sample | Concentration | Harvest Viscosity |
| (vessel) | (g/L) | (cP) |
| MK29404 WT | 22.78 | 1700 |
| (10 L) | | |
| MK29404 Dry-1 | 5.38 | 8.0 |
| (10 L) | | |
| MK29404 WT | 2.81 | 4.11 |
| Shaker Flask | | |
| MK29404 Dry-1 | 0.68 | 1.68 |
| Shaker Flask | | |

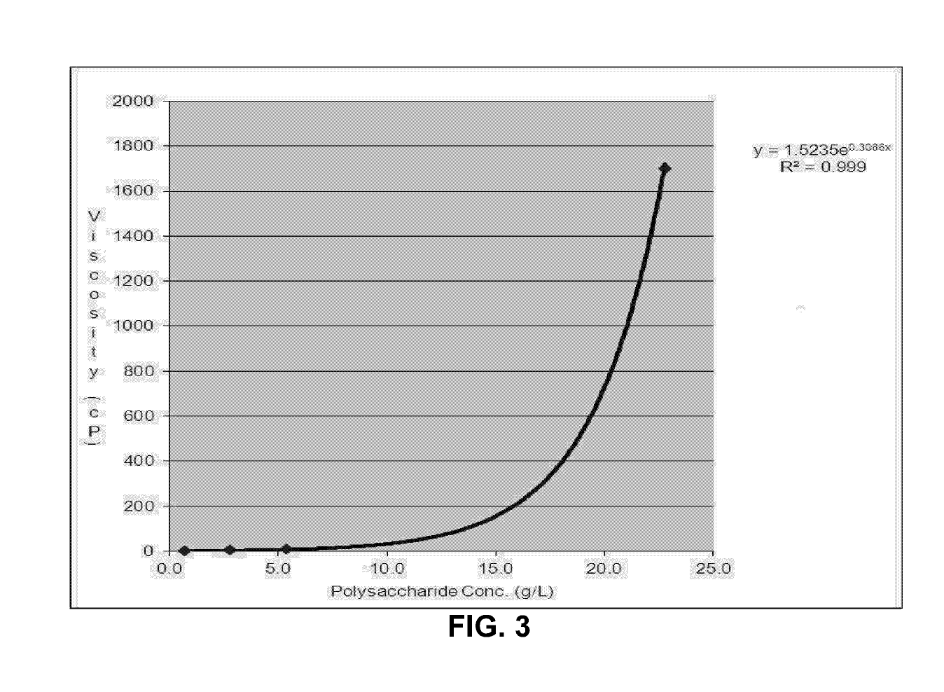

The observed viscosity of the solutions was plotted as a function of concentration of polysaccharide in solution. The graph of this correlation is shown in FIG. 3. The correlation is as follows:

Viscosity=1.5*e^{0.30*polysaccharide concentration}

This empirical correlation shows that viscosity increases exponentially with increasing polysaccharide concentration. This result indicates that reducing the polysaccharide concentration will exponentially decrease solution viscosity, and in turn dramatically decrease the power per volume required to deliver oxygen.

For both the high and low volume fermentations, the MK29404 WT strain produces about at least 4 times the amount of polysaccharide than the MK29404 dry mutant strain (10 L high volume: 4.23 times, Shaker flask low volume: 4.13 times). (Table 3) This suggests that the low volume shaker flask experiments are representative of each strain's polysaccharide production in the large volume fermentation. Thus, the low volume shaker flasks can be used as an accurate and efficient model to study the effects of polysaccharides and viscosity in Dry mutants.

TABLE 4
Summary at Max % Lipid, ratio calculations

| | | Non-RV | | | | | |
|---|---|---|---|---|---|---|---|
| | FA | Cell | Non-RV | | | | PS to |
| | Yield | Density | Fat (g/L) | | Poly- | PS to | TOTAL |
| | on | (g/L) | (TOTAL | | Saccharide | Lipid | BIOMASS |
| | sugar | (TOTAL | LIPID | % Fat | Conc. | RATIO | RATIO |
| Strain | (wt %) | MASS) | MASS) | (FAME) | (g/L) | (g/g) | (g/g) |
| MK29404 WT | 15.21 | 116.05 | 58.05 | 50.02 | 22.78 | 0.0039 | 0.20 |
| (standard media) | | | | | | | |
| MK29404 (Dry1) | 19.99 | 127.36 | 75.56 | 59.32 | 5.38 | 0.0007 | 0.04 |
| (Riceland media) | | | | | | | |

### Example 9

### Determining Exocellular Polysaccharide Composition

The monosaccharide composition of the exocellular polysaccharide produced by MK29404 Dry-1 was analyzed. The MK29404 wild type polysaccharide was also assayed to determine whether any structural differences existed between the Dry and WT strains.

Strains were grown under high volume 10 L fermentation conditions and in low volume shaker flask conditions, as described above. Polysaccharides were isolated as described from both WT and Dry mutant strains under both fermentation conditions. The isolated polysaccharides were depolymerized to determine the quantity of monosaccharide components. This was done using acid hydrolysis of the polysaccharide, described in detail in U.S. Pat. No. 4,664,717; Hoebler, et al. J. Agric. Food Chem., 37:360-367 (1989), which are incorporated by reference.

Briefly, a small sample of crude polysaccharide is placed into centrifuge tube. 5 mL of 2N HCl is dispensed into the tube with sample and placed in 60 degree Celsius water bath, as the sample will not dissolve at room temperature. The sample is vortexed frequently in the warm water bath until the sample has completely dissolved. Once dissolved, the sample solution is incubated at 60 degree Celsius for at least 2 hours, After 2 hours, the sample is removed from the water bath and allowed to cool to room temperature, and diluted as necessary. Ion exchange chromatography (IEC) is then used to analyze the sample using a Carbopac SA10 column. The IEC chromatograms for the depolymerized MK29404 WT polysaccharide is shown in FIG. 4. The IEC chromatograms for the depolymerized MK29404 Dry-1 mutant polysaccharide is shown in FIG. 5. As can be seen, the IEC chromatograms have different retention times, suggesting a difference in monosaccharide composition of the polysaccharides produced by each strain.

The stoichiometric composition of each depolymerized polysaccharide sample can then be quantified using the appropriate standards. See for example Dubois, M., et al. Anal. Chem. 28:350-356 (1956), and U.S. Pat. No. 5,512,488. Briefly, the crude polysaccharide is weighed and diluted with deionized water until complete dilution. 0.5 mL of the crude polysaccharide is transferred to a tube containing 0.5 mL of a 4% (w/v) phenol solution and vortexed. 2.5 mL of concentrated sulfuric acid solution is then added and vortexed. The solution is then allowed to cool to room temperature, and the absorbance at 490 nm is measured. This absorbance correlates with the color of the polysaccharide. The sample is then diluted as necessary, and a stock standard is prepared using the same stoichiometric proportions of monosaccharides as found in the sample. The approximate total polysaccharide concentration in the initial aliquot can then be calculated by multiplying the purity factor by the net polysaccharide mass obtained from isolation procedure.

The results for the 10 L fermentation are shown in Table 5. The monosaccharide composition of the polysaccharides for the low volume shaker flask fermentations are shown in Table 6. Identifying specific polysaccharides are not possible from data.

TABLE 5
Monosaccharide composition after acid hydrolysis of
strains grown in 10 L fermentors

| | | Wt. Area | Molar |
|---|---|---|---|
| Sample | Monosaccharide | (%) | (%) |
| MK29404 | Fucose | 10.65 | 11.28 |
| WT | Arabinose | 0.86 | 0.99 |
| | Galactose | 30.69 | 29.62 |
| | Glucose | 26.80 | 25.87 |
| | Xylose | 11.91 | 13.80 |
| | Mannose | 0.44 | 0.42 |
| | Fructose | 18.66 | 18.02 |
| MK29404 | Fucose | 20.18 | 21.20 |
| Dry-1 | Arabinose | 10.64 | 12.22 |
| | Galactose | 47.78 | 45.74 |
| | Glucose | 5.46 | 5.23 |
| | Xylose | 1.76 | 2.02 |
| | Mannose | ND | ND |
| | Fructose | 14.19 | 13.59 |

TABLE 6
Monosaccharide composition after acid hydrolysis
of strains grown in shaker flasks

| | | Concentration | Molar |
|---|---|---|---|
| Sample | Monosaccharide | (mg/g PPT) | (%) |
| MK29404 | Fucose | 3.89 | 2.41 |
| WT | Arabinose | ND | ND |
| | Galactose | 42.26 | 23.85 |
| | Glucose | 16.05 | 9.06 |
| | Xylose | 0.10 | 0.07 |
| | Mannose | 111.59 | 62.97 |
| | Fructose | 2.93 | 1.65 |
| | Sucrose | ND | ND |
| MK29404 | Fucose | 1.34 | 4.82 |
| Dry-1 | Arabinose | ND | ND |
| | Galactose | 16.87 | 94.42 |
| | Glucose | 0.14 | 0.76 |
| | Xylose | ND | ND |
| | Mannose | ND | ND |
| | Fructose | ND | ND |

### Example 10

### Size Exclusion Chromotography of Isolated Polysaccharides

The isolated polysaccharides produced by MK29404 Dry-1 and MK29404 WT were analyzed by size exclusion chromatography (SEC). SEC of polysaccharides is described in detail in Hoagland, et al., J. Agricultural and Food Chem. 41(8): 1274-1281 (1993). Briefly, the various polysaccharides produced by each of the strains will separate according to molecular weight, exposing any differences between the polysaccharides produced by the WT versus the Dry-1 mutant.

The SEC was run using a column with an exclusion limit of 300 kD. A representative SEC readout overlaying the MK29404 Dry-1 and WT polysaccharides is shown in FIG. 6. The readout shows that MK29404 WT contains polysaccharides of higher MW (≧300 kD) in greater relative abundance than MK29404 Dry-1.

What is claimed is:

1. An oleaginous microorganism suitable for production of renewable materials, wherein the microorganism comprises a genetic modification not present in an unmodified microorganism, and wherein the modified microorganism produces a fermentation broth having a lower viscosity than a fermentation broth produced by the unmodified microorganism when grown in culture.

2. The oleaginous microorganism of claim 1, wherein the modified microorganism produces a fermentation broth comprising a biomass of at least about 50 grams cellular dry weight per liter and a viscosity of less than about 1,100 centipoise (cP).

3. The oleaginous microorganism of claim 1, wherein the modified microorganism produces a fermentation broth comprising a biomass of at least about 50 grams cellular dry weight per liter and a viscosity of less than about 30 cP.

4. The oleaginous microorganism of claim 1, wherein the modified microorganism comprises a dry morphology, while the unmodified microorganism does not comprise a dry morphology.

5. The oleaginous microorganism of claim 1, the microorganism being the microorganism corresponding to one or more of ATCC Deposit No. PTA-12508 (Strain MK29404 (Dry1-13J)), ATCC Deposit No. PTA-12509 (Strain MK29404 (Dry1-182J)), ATCC Deposit No. PTA-12510 (Strain MK29404 (Dry1-173N)), ATCC Deposit No. PTA-12511 (Strain MK29404 (Dry55)), ATCC Deposit No. PTA-12512 (Strain MK29404 (Dry41)), ATCC Deposit No. PTA-12513 (Strain MK29404 (Dry1)), ATCC Deposit No. PTA-12515 (Strain MK29404 (Dry1-147D)), or ATCC Deposit No. PTA-12516 (Strain MK29404 (Dry1-72D)).

6. The oleaginous microorganism of claim 1, the microorganism being the microorganism corresponding to one or more of ATCC Deposit No. PTA-12506 (Strain MK29794 (KDry16-1)), ATCC Deposit No. PTA-12507 (Strain MK29794 (KDry7)), ATCC Deposit No. PTA-12514 (Strain MK29794 (K200 Dry1)), or ATCC Deposit No. PTA-12517 (Strain MK29794 (33 Dry1)).

7. The oleaginous microorganism of claim 1, wherein the modified microorganism produces a fermentation broth comprising a biomass of at least about 50 grams cellular dry weight per liter and a viscosity at least about 10 times lower than the viscosity of a substantially similar fermentation broth produced by the unmodified microorganism.

8. The oleaginous microorganism of claim 1, wherein the modified microorganism and the unmodified microorganism produce a fermentation broth comprising an exocellular polysaccharide.

9. The oleaginous microorganism of claim 1, wherein the modified microorganism produces at least about 2 times less exocellular polysaccharide per liter of fermentation broth than the unmodified microorganism.

10. The oleaginous microorganism of claim 1, wherein the modified microorganism has a dry weight as fatty acids of at least about 25 percent.

11. The oleaginous microorganism of claim 1, wherein the modified microorganism can be cultured in fermentation broth requiring less than 8.0 horsepower per 1000 gallons for agitation.

12. A fermentation broth produced by the modified microorganism claim 1.

13. The fermentation broth of claim 12, wherein the fermentation broth comprises a lipid to exocellular polysaccharide ratio of greater than about 2.

14. A method of producing a biofuel precursor, the method comprising culturing the microorganism of claim 1 and collecting the fermentation broth produced by the microorganism.

15. A method of producing a biofuel, the method comprising:
(a) supplying a carbon source;
(b) converting the carbon source to fatty acids within the microorganism of claim 1;
(c) culturing the microorganism to a cell density of at least about 50 grams cellular dry weight per liter in a fermentation broth having a viscosity of less than about 1100 cP;
(d) extracting fatty acids from the microorganism; and
(e) reacting the fatty acids to produce a biofuel.

16. The method of claim 15, the microorganism being an exocellular polysaccharide-producing yeast.

17. A biofuel produced by the method of claim 15.

18. A method of powering a vehicle by combusting the biofuel claim 17 in an internal combustion engine.
